(19) United States
(12) Patent Application Publication
(10) Pub. No.: US 2025/0152243 A1
(43) Pub. Date: May 15, 2025

(54) PROCESS AND SYSTEM FOR THREE-DIMENSIONAL MODELLING OF TISSUE OF A SUBJECT, AND SURGICAL PLANNING PROCESS AND SYSTEM

(71) Applicant: CONOVA MEDICAL TECHNOLOGY LIMITED, Hong Kong (CN)

(72) Inventor: Morgan Mao CHENG, Hong Kong (CN)

(73) Assignee: CONOVA MEDICAL TECHNOLOGY LIMITED, Hong Kong (CN)

(21) Appl. No.: 18/834,607

(22) PCT Filed: Jan. 31, 2023
(86) PCT No.: PCT/CN2023/074042

§ 371 Date: Jul. 30, 2024

(30) Foreign Application Priority Data
Jan. 31, 2022 (HK) 32022047546.3
Mar. 11, 2022 (HK) 32022049866.3

(51) Int. Cl. A61B 34/10

(52) U.S. Cl. CPC A61B 34/10; A61B 2034/107

(57) ABSTRACT

A process for tissue modelling from a medical image of a subject, for forming a three-dimensional (3D) model of a region of interest (ROI) of a subject with one or more tissue types, said process including the steps of (i) utilising a rule-based method that automatically generates the weak annotation, initial seed area from a medial image (210b); (ii) utilising a proposal generation method that integrates the multi-scale image features and anatomical prior (220b); and (iii) a comprehensive loss for CNN training that optimizes the pixel classification and feature distribution simultaneously (230).

### TECHNICAL FIELD

The present invention relates to a process and system for generation of a three-dimensional model of one or more tissue-types in the region of interest of a subject and for surgical planning, and in particular the present invention relates to a process and system for bone surgical planning for orthopaedic procedures.

### BACKGROUND OF THE INVENTION

As is known, surgical planning is a pre-operative, and in some cases intra-operative step, which indispensable step in surgery to detect pathology and avoid potential risks.

Furthermore, surgical planning is advantageous in the orthopaedic discipline, in order to determine implant type, size and location in some cases.

Also as is known, the accuracy of surgical planning greatly affects the outcome of surgical treatment and procedures.

Currently, surgical planning is typically conducted manually by a surgeon based on computerized tomography (CT) and Magnetic Resonance Imaging (MRI) images of a subject.

Due to the high complexity of the planning process for surgical procedures, manual surgical planning process typically takes a relatively long time, which reduces the efficiency of treatment of the subject.

Furthermore, manual planning inevitably includes a subjective factor of manual assessment, the inter-rater variations are inevitable, which may influence the success rate of surgical treatment and procedures.

### OBJECT OF THE INVENTION

It is an object of the present invention to provide a process and system for surgical planning for which overcome or at least partly ameliorate at least some deficiencies as associated with the prior art.

### SUMMARY OF THE INVENTION

The present inventor has identified the shortcomings of the existing techniques, and has sought to provide a surgical planning process and system, which addresses the deficiencies associated with the prior art, in particular in relation to use of CT images, for reasons as identified, which is the lack of ability to provide for surgical planning of procedures involving soft tissue, in particular surgical procedures relating to orthopaedics and more particularly spinal surgery procedures.

Thus, the present invention is directed to providing a system for multi-tissue type for 3D modelling for surgical planning and analysis.

In a first the present invention provides process for tissue modelling from a medical image of a subject, for forming a three-dimensional (3D) model of a region of interest (ROI) of a subject with one or more tissue types, said process including the steps of:
(i) utilising a rule-based method that automatically generates the weak annotation, initial seed area from a medial image;
(ii) utilising a proposal generation method that integrates the multi-scale image features and anatomical prior; and
(iii) a comprehensive loss for Convolutional Neural Network (CNN) training that optimizes the pixel classification and feature distribution simultaneously.

In a second aspect, the present invention provides a process for tissue modelling of a subject of one or more tissue type at a region of interest (ROI) of said subject, for forming a three-dimensional (3D) model of a region of interest (ROI) of a subject with one or more tissue types, said process including the steps of:
(i) utilising a rule-based method to automatically generate a weak annotation from a midline slice of a subject in a 3 Dimensional (3D) medical image of said subject, wherein the rule-based method detects the approximate tissue locations from the midline slice, and, and further determines the initial seed areas;
(ii) developing a neural network model to generate multi-scale feature maps and pixel classifications from the midline slice;
(iii) utilising a clustering-based method to generate segmentation proposals based on multi-scale feature maps and the seed areas from (i) and (ii).
(iv) further fine-tuning the proposal with several rule-based operations to explicitly embed within an anatomical prior of the region of interest (ROI), and wherein the seed areas are updated according to the fine-tuned proposal; and
(v) training the neural network model with a comprehensive loss, which simultaneously optimizes the pixel classification and feature distribution of feature maps based on the proposals.

The process may provide for modelling of multiple tissue type of a subject in the region of interest.

The medical image may be a Magnetic Resonance Imaging (MRI) image.

The process may further include the steps of:
utilising one of more further slices of said subject acquired of the region of interest (ROI) of the subject at varying depths within the region of interest (ROI) and simultaneously optimizes the pixel classification and feature distribution of feature maps based on the proposals for said one or more further slices by the process of the second aspect steps (i) to (ii); and
forming a three dimensional (3D) model of the region of interest (ROI) of the tissue of the subject from the midline slice and the one or more further slices.

In a third aspect, the present invention provides a system for tissue modelling of a subject of one or more tissue type at a region of interest (ROI) of said subject, for forming a three-dimensional (3D) model of a region of interest (ROI) of a subject with one or more tissue types, said system including an input module, a processor, a neutral network, and an output module, wherein:
said input module receives a 3 Dimensional (3D) medical image containing a plurality of 2 Dimensional (2D) slices of a subject of a region of interest (ROI) of said subject;
said processor and a neural network provide the process of:
(i) using a rule-based method to automatically generate a weak annotation from the plurality of 2 Dimensional (2D) slices of said subject, wherein the rule-based method detects the approximate tissue locations from the plurality of 2 Dimensional (2D) slices, and further determines the initial seed areas;
(ii) developing a neural network model to generate multi-scale feature maps and pixel classifications from the plurality of 2 Dimensional (2D) slices;
(iii) utilising a clustering-based method to generate segmentation proposals based on multi-scale feature maps and the seed areas;
(iv) further fine-tuning the proposal with several rule-based operations to explicitly embed within an anatomical prior of the region of interest (ROI), and wherein the seed areas are updated according to the fine-tuned proposal; and
(v) training the neural network model with a comprehensive loss, which simultaneously optimizes the pixel classification and feature distribution of feature maps based on the proposals; and
said output module provides an output representation a three-dimensional (3D) model of a region of interest (ROI) of the subject with one or more tissue types.

In a fourth aspect, the present invention provides a three-dimensional (3D) medical model of a region of interest of a subject, wherein the three-dimensional medical model is formed by way of the process of the second aspect and further include the steps of utilising one of more further slices of said subject acquired of the region of interest (ROI) of the subject at varying depths within the region of interest (ROI) and simultaneously optimizes the pixel classification and feature distribution of feature maps based on the proposals for said one or more further slices by the process of the second aspect steps (i) to (ii); and forming a three dimensional (3D) model of the region of interest (ROI) of the tissue of the subject from the midline slice and the one or more further slices.

In a fifth aspect, the present invention provides a process for providing a three-dimensional (3D) tissue model of one or more tissues or tissue types, the process including the steps of:
(i) providing unique rule for a seed, that is a portion of an analytical site which is used to start, in a medical image;
(ii) using the seed area to determine a segmentation proposal and
(iii) training a deep learning model with the segmentation proposal.

### PRESENT INVENTION

In accordance with the present invention, in order to provide a multi-tissue three-dimensional (3D) model of tissue of a subject however, the present invention is also applicable to single tissue structures for analysis.

Medical images may be utilised, and preferably Magnetic resonance images (MRIs) are utilised, as such images can simultaneously illustrate the 3D structures and potential pathologies of multiple tissue types and regions. However, in some cases, CT or other types of medical images can be utilised in the present invention.

Further, although the present invention is applicable to multi-tissue 3D modelling, in some cases the present invention may be used for single tissue or tissue type.

In accordance with the present invention, there is provided a process for providing a 3D dimensional tissue model of one or more tissues or tissue types, the process including the steps of:
(i) Providing unique rule for a seed, that is a portion of an analytical site which is used to start, in a medical image; and
(ii) Using the seed area to determine a segmentation proposal.
(iii) Training a deep learning model with the segmentation proposal.

### Process

In general terms, the process is a seed+feedback arrangement and can, in some embodiments, include both pre and post processing in addition to the general algorithm and process provided.

### Implementations

The present invention can be implemented by way of a system in a hospital clinical environment, in the “cloud” by way of external server, by way of local server.

### Advantages

The present invention provides advantages over those of the prior art, including:
(a) Low capital power required,
(b) No need for quantum computer power or usage,
(c) Obviates human inter and intra variance, and
(d) Not scanning machine dependent, and can be utilised with outputs from various types of scanning machines.
(e) No human annotation is required.

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings.

The drawings presented herein may not be drawn to scale and any reference to dimensions in the drawings or the following description is specific to the embodiments disclosed, in which:

FIG. 1A and FIG. 1B show an example of a sagittal lumbar MRI;

FIG. 1C and FIG. 1D show examples of image feature variation of MRI;

FIG. 2A shows an exemplary embodiment of a process of the present invention;

FIG. 2B shows a further exemplary embodiment of a process of the present invention for tissue modelling from a medical image of a subject;

FIG. 2C shows another exemplary embodiment of a process of the present invention for tissue modelling of a subject;

FIG. 2D shows an exemplary embodiment of a system of the present invention for tissue modelling of a subject of one or more tissue type at a region of interest (ROI);

FIG. 2E shows an exemplary embodiment presents the process of the present invention;

FIG. 2F shows for each tissue type, the initial seed area consists of small 3D neighbourhoods around the tissue locations, which are not necessarily in the same slice due to potential scoliosis or other distortions or inconsistencies in other ROIs and tissue, for example, in accordance with the present invention;

FIG. 3A-3H shows a rule-based seed area initialization;

FIG. 4A and FIG. 4B present an MRI patch and one of its feature maps generated by the Computer Neural Network (CNN) model;

FIG. 4C shows the clustering result of the feature map;

FIG. 4D shows the pixel division based on the clustering result;

FIG. 5 shows a pixel selection process;

FIG. 6 shows a seed area updating;

FIG. 7 shows a CNN model and comprehensive loss;

FIG. 8A and FIG. 8B shows the initial seed areas and multi-tissue segmentations respectively;

FIG. 8C to FIG. 8F shows the segmentation on MRI patches produced by different methods;

FIG. 9 shows evolution of segmentation performance (mean Dice) during the HT process;

FIG. 10 shows the proposals generated by Spine-GFlow (P−) and standard Spine-GFlow in different iterations;

FIG. 11 shows the pixel clustering results of feature maps generated by the CNN model of Spine-GFlow;

FIG. 12A and FIG. 12B show two examples of rule-based fine-tuning that showed fine-tuning could fill the cavity as in FIG. 12A and remove error pixels as in FIG. 12B in the proposals;

FIG. 13 shows the proposals and segmentation results produced with the defective initial seed areas;

FIG. 14 shows a schematic representation of an example for use with the present invention;

FIG. 15 shows a schematic representation of an example for use with the present invention of an Artificial Intelligence (AI) client;

FIG. 16 shows a schematic representation of an example for use with the present invention of an AI server;

FIG. 17 shows a schematic representation of procedure of rule-based seed area initialization according to the present invention;

FIG. 18 shows a schematic representation of an example of a procedure of segmentation proposal generation according to the present invention;

FIG. 19 shows a schematic representation of an example of network architecture of AI model for segmentation according to the present invention;

FIG. 20 shows a schematic representation of an example of a network architecture of AI model for slice super-resolution according to the present invention;

FIG. 21 shows a schematic representation of an example of a framework of Storage of AI system, according to the present invention;

FIG. 22 shows a schematic representation of an example of a framework of Augmented Reality (AR) system, according to the present invention; and

FIG. 23 shows a schematic representation of an example of a deployment scenarios, according to the present invention.

### DETAILED DESCRIPTION OF THE DRAWINGS

The present inventors have identified shortcomings in processes and systems of the prior art, and upon identification of the problems with the prior art, have provided a process and system which overcomes the problems of the prior art.

### 1. BACKGROUND—PRIOR ART AND DEFICIENCIES

As identified by the present inventor, several 3D reconstruction techniques based on 3D medical images have been developed. Most 3D reconstruction techniques are based on the CT images, because of the high resolution and contrast which is provided.

However, as noted by the present inventor, for some types of surgery which involve both bone and soft tissue, such as spine surgery, since a CT image only presents the bone structural material, these techniques can only provide the 3D reconstruction of vertebrae.

As is known and noted by the present inventor, soft tissue, such as nerves and blood vessels, are extremely important in the surgical planning. Any accidental damage to these types of soft tissues during surgery cause great irreversible damage to the patient.

The present inventor has noted that existing techniques can only assist surgeon in surgical planning on the bone structure and not soft tissue. Accordingly. during a surgical process, the surgeon's experience is required to avoid damage of important soft tissue, which inherently has large risks associated with it.

Within the prior art, as identified by the present inventor, planning and analysis is typically done via (i) traditional rules-based methodology and (ii) manual labelling/leaning methods, and both of which may be implemented into computerised methodologies.

Accordingly, the present inventor has identified and noted deficiencies with the prior art.

### 2. BACKGROUND OF PRESENT INVENTION

As is known, within most learning-based magnetic resonance image (MRI) segmentation methods rely on the manual annotation to provide supervision, which can be considered to be extremely tedious, especially when multiple anatomical structures are required.

In order to address and overcome deficiencies of the prior art, this work, the present inventor has developed a hybrid framework, referred to herein as “Spine-GFlow” with reference to the present invention, that combines image features learned by a Convolutional Neural Network (CNN) model and anatomical priors for multi-tissue segmentation in a sagittal lumbar MRI.

As will be understood, the term “Spine-GFlow” refers to the example of the implementation of the process and system according to the present invention, in particular with reference to spinal applications as shown in the following examples and comparative analysis of the present invention in respect of the prior art.

It should be understood and appreciated, that although the present example is directed towards multi-tissue segmentation in sagittal lumbar MRI, the invention is also applicable full analysis of single tissue, such as blood vessels alone, in other organs or associated there with, as well as in the spine,

It should be understood and appreciated, that the present invention and also may utilise medical images other than MRI, for example CT scan images.

Advantageously and notably, the present invention does not require any manual annotation and is robust against image feature variation caused by different image settings and/or underlying pathology.

As such, importantly and advantageously, the present invention may be considered to be machine independent and have greater versatility.

### 3. GENERAL CONCEPT OF PRESENT INVENTION SUMMARY AND STUDIES

The present invention includes:
(i) a rule-based method that automatically generates the weak annotation (initial seed area);
(ii) a novel proposal generation method that integrates the multi-scale image features and anatomical prior; and
(iii) a comprehensive loss for CNN training that optimizes the pixel classification and feature distribution simultaneously.

The present invention has been validated on 2 independent datasets: Hong Kong Disc Degeneration Cohort (HKDDC) containing images obtained from 3 different machines) and Intervertebral Disc Localization and Segmentation (IVDM3Seg).

The segmentation results of vertebral bodies (VB), intervertebral discs (IVD), and spinal canal (SC) were evaluated quantitatively using Intersection over Union (IoU) and the Dice coefficient.

Results showed that the process of the present invention, without requiring manual annotation, has achieved a segmentation performance comparable to a model trained with full supervision (mean Dice 0.914 vs 0.916).

### 4. BACKGROUND ART AND OBSERVATIONS BY PRESENT INVENTOR

As is known, Magnetic Resonance Images (MRIs) are widely used in the clinic for the diagnosis of degenerative lumbar disease. (Benneker et al., 2005; Cheung et al., 2019; Jensen et al., 1994; Lai et al., 2021a; Lai et al., 2021b; Pfirrmann et al., 2001).

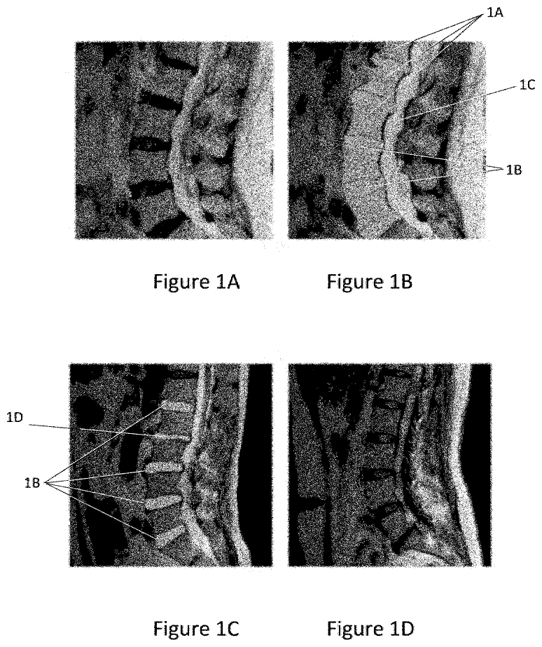

As an MRI allows the visualization of the 3D structure of soft tissues including intervertebral discs (IVD) and the spinal canal (SC) as is shown in FIG. 1A and FIG. 1B and is considered the gold standard for the assessment of IVD herniation (Benneker et al., 2005; Pfirrmann et al., 2001) and spinal stenosis (Cheung et al., 2019; Lai et al., 2021a; Lai et al., 2021b).

Currently, analysis of lumbar MRIs relies heavily on the experience and subjective judgment by specialist clinicians, which makes the assessment process laborious and potentially inaccurate with inevitable inter-rater variations, as will be readily understood.

Thus, as noted by the present inventor, automated and objective lumbar MRI assessments are highly desirable.

Semantic segmentation is considered to be important for auto-analysis of lumbar MRIs as it provides the locations and pixel-wise anatomical information of spinal tissues, which serve as precursors for further pathology and disease progression predictions.

Conventional semantic segmentation methods for lumbar MRI are rule-based and based on graphical and anatomical priors of target tissue (Carballido-Gamio et al., 2004; Egger et al., 2012; He et al., 2017; Michopoulou et al., 2009; Neubert et al., 2012).

Pre-determined templates, detectors, and rules are manually designed for the segmentation task.

However, these rule-based methods are not sufficiently robust against the highly variable image features in MRI caused by systematic and/or individual deviations (Cheng and Halchenko, 2020). The systematic deviation is usually caused by different MRI protocols, equipment settings, and human operations, which are common when MRI images are obtained from different institutions.

The individual deviation is usually caused by underlying pathologies, such as shape and alignment deformity, which are random, and which can vary widely between individuals. Several examples of image feature variation as shown in FIG. 1C and FIG. 1D, including shape distortion, low pixel intensity, low contrast, unclear edges, and noise, can be observed.

These rule-based methods of the prior art can detect approximate tissue locations but have been noted by the present inventor to often fail to obtain accurate shape information. As a result, they are considered not suitable to be used directly in clinical practice.

Furthermore, these rule-based methods are usually designed based on specific tissue, thus as noted by the present inventor, disadvantageously they can only segment a single tissue.

As noted by the present inventor, multi-tissue segmentation is important considering that clinical diagnosis often requires a comprehensive analysis of multiple tissues.

As is shown in FIG. 1A and FIG. 1B, there is an example of a sagittal lumbar MRI that clearly shows multiple spinal tissues including vertebral bodies 1A, intervertebral discs 1A, and the spinal canal 1C.

FIG. 1C illustrates serious shape distortion of an intervertebral disc 1D due to disc degeneration. FIG. 1D presents an MRI with low image quality including low pixel intensity, low contrast, unclear edges, and noise.

As is noted, the rapid development of convolutional neural networks (CNN), learning-based methods have achieved remarkable performance in semantic segmentation.

For medical images, a CNN model trained with full pixel-wise annotation (termed “full-supervision”) can obtain accuracy comparable to clinical specialists.

However, as noted by the present inventor, the required manual annotation is extremely laborious and time-consuming, which makes full-supervision costly and large-scale annotated datasets very scarce.

To address this limitation, weakly-supervised methods have been developed in the prior art.

Such weakly-supervised methods train models with weak annotations which can significantly reduce the cost of full-supervision; priors of tissues such as pixel value, shape, and size are usually utilised to support training.

As noted by the present inventor, nonetheless, for 3D images such as MRI and CT of the prior art, weak annotation is still very expensive and time consuming, as each slice needs to be annotated separately.

Furthermore, as noted by the present inventor, as the CNN model of such prior art processes is data-sensitive and vulnerable to the variation of image features, disadvantageously new annotations may be required to fine-tune the model for images acquired under different settings.

Furthermore, as noted by the present inventor, such a well-trained model may also fail in the event the case with underlying pathology.

### 5. PRESENT INVENTION

In the present invention, there is combined rule-based and learning-based methods, and a hybrid framework for multi-tissue segmentation for clinical analysis, which in the present example is for spinal analysis application using MRI as an imaging technique, thus in lumbar MRI that requires no manual annotation.

A rule-based method is designed to automatically generate the incomplete (within a few MRI slices) and inaccurate (missing and location deviation) weak annotation.

In the process of the present invention, the rule-based method first identifies approximate tissue locations and in the case of spinal analysis a rough spinal region, and further determines the initial seed areas.

An iterative optimization procedure is then utilised to train a Convolutional Neural Network (CNN) model with the initial seed areas.

The CNN model can generate multi-scale feature maps and pixel classification from MRI images.

The optimization procedure iterates between two steps:
(i) proposal generation; and
(ii) CNN training.

In proposal generation, the multi-level information is integrated within the multi-scale feature maps to produce the segmentation proposals based on the seed areas.

The rule-based proposal fine-tuning is adopted to explicitly embed the anatomical prior.

In CNN training, a comprehensive loss is adopted to optimize the pixel classification and feature distribution of feature maps simultaneously based on the proposals.

It is considered that with the iterative optimization procedure, the framework of the present invention can gradually optimize the proposals and CNN model, and the optimized CNN model can produce accurate multi-tissue segmentation, for example in the lumbar MRI.

Advantageously, as no manual annotation is required in the framework of the present invention, it can automatically fine-tune the CNN model on the target MRI, which can effectively improve the robustness of the model against image feature variation caused by different image settings and/or underlying pathology.

Unlike other unsupervised segmentation methods of the prior art that do not use any annotation in the training process, the present invention framework utilises automatic annotation, which can guide the model to generate more semantic features, advantageously rather than focusing on the shallow image features.

Thus, the present invention provides a hybrid framework, an embodiment of which has been termed “Spine-GFlow” which refer to implementations of the process and systems of the present invention, for the robust segmentation of multiple tissues including vertebral bodies (VB), IVD, and SC in sagittal lumbar MRI images without relying on any manual annotation or human intervention.

It should be noted that the term “Spine-GFlow” name is derived because (i) this framework is specifically tuned based on the anatomical knowledge of the spine, which is a complex organ consisting of multiple types of tissues; and (ii) “G” stands for “Generative” as advantageously manual annotations are not required but generating masks automatically.

The objectives which include:
1) designing a rule-based method that automatically generates the weak annotation (initial seed area);
2) developing a proposal generation method that integrates the multi-scale feature maps and anatomical prior; and
3) training the CNN model with a comprehensive loss that optimizes the pixel classification and feature distribution of feature maps simultaneously,

Advantageously, the present invention provides a system and process which can be supported and implemented on such any system whereby the system can be within a hospital, within a cloud computing environment, a local server, or combinations thereof.

Again, although examples and description of the invention as provided in particular in reference to the spinal anatomy, which includes multiple tissues, including bone, soft tissue, vertebrate, nerves and blood vessels, it must be appreciated and understood that the present invention is equally applicable to other tissue environments in the body of a subject or an animal and for the implementation of analysis of tissue. Thus, the term “Spine-GFlow” must not be considered or interpreted as limiting the present invention to spinal applications.

Advantageously, the present invention can be implemented for the imaging and surgical planning in relation to tissue structures hold the body of a human or an animal, and provides an efficient and advantageously time and cost effective solution in view of the prior art form providing images of multiple tissue portions of the body for surgical planning.

Also, although the present invention is described particularly in relation to multiple tissue analysis and imaging, for surgical planning, the present invention is also applicable for single tissue analysis, for example the blood vessels or nerves of the spine or the subject in need of investigation, analysis or surgical intervention.

Furthermore, although the present invention is described in reference particularly to MRI type images for medical imaging in the illustrative example, as will be understood by those skilled in the art, other imaging techniques may be also implemented within the present invention, such as CT (computed tomography) or NMR (Nuclear Magnetic Resonance) imaging, and that the present invention is not limited or restricted to any particular type of medical imaging.

Advantageously, the present invention is versatile and does not require excessive capital input, is time and cost efficient in analysis obviates human enter and intra variance, problems as identified by the present inventor in respect of techniques of and processes as provided by the prior art.

Furthermore and advantageously, the present invention provides a process and system which is independent of the machine on which the scanning or acquisition of the medical image has been performed. It is not reliant on particular processing and thus provides machine independent analysis, for providing images from various imaging machines and types and brands, and thus, provides a more efficient and cost effective and time effective solution to medical imaging in comparison with those as provided by the prior art.

### 6. PRIOR ART RELATED PROCESSING AND WORKS

### 6.1 Rule-Based MRI Segmentation—Example Spine/Lumbar

Rule-based segmentation methods, in particular for spine/lumbar MRI, are typically developed based on the graphical or anatomical priors of specific tissues.

Normalized cut (NCut) has been adopted by Carballido-Gamio et al., 2004, to segment Vertebral Bodies (VBs) from midline sagittal spine MRIs. A multi-feature and adaptive spectral segmentation were proposed by He et al., 2017 to segment spinal neural foramina within preselected Region of Interest (ROIs).

A statistics-based method was proposed by Neubert et al., 2012 to segment Intervertebral discs (IVD) and VB with statistical shape analysis and registration of grey level intensity profiles.

An atlas-based segmentation method for IVD that relied on manually-designed templates was proposed by Michopoulou et al., 2009. Shape information was utilized by Egger et al., 2012 to produce the segmentation of VB, which relied on manually selected seed points for initialization.

It has been noted by the present inventor, that disadvantageously all rule-based methods above (i) can only produce the segmentation of one kind of tissue at a time, and (ii) modification was required to transfer these methods to different tissues.

Additionally, and disadvantageously, some methods required human intervention to guide segmentation such Egger et al., 2012 and He et al., 2017.

### 6.2 Weakly-Supervised Segmentation Per Se

The training a model for a segmentation task with weak annotations such as (a) image tag (Pathak et al., 2015), (b) bounding boxes (Dai et al., 2015; Khoreva et al., 2017; Kulharia et al., 2020; Lee et al., 2021; Song et al., 2019), (c) scribbles (Lin et al., 2016; Tang et al., 2018), and (d) points (Bearman et al., 2016) has been considered an attractive problem.

As note by the present inventor, a key idea for weakly-supervised segmentation is to integrate the priors about the object, for example shape, size, relative location and the like.) and image parameters such as colour, texture, brightness and the like, in the training process.

BoxSup by Dai et al., 2015 proposed an iterative procedure that iterates between proposal generation and model training to gradually improve the proposals and the model.

Other previous work (Khoreva et al., 2017) demonstrated that with a carefully designed proposal, the model could achieve better performance with much fewer training rounds.

Attention mechanism was applied (Kulharia et al., 2020; Song et al., 2019) to guide the model to focus on specific areas of objects in the image.

Pixel-embedding learning was adopted (Kulharia et al., 2020) to generate pixel features with high intra-class affinity and inter-class discrimination. Priors of objectness filling rates were adopted (Song et al., 2019) to support training.

The BBAM (Lee et al., 2021) utilised higher-level information to identify small informative areas in the image, which served as a pseudo-ground-truth for training the segmentation model.

The CCNN (Pathak et al., 2015) adopted a constrained loss to integrate the priors in the training process, which imposed linear constraints on a latent distribution of the model output and trained the model to be close to the latent distribution.

A generic objective prior was directly incorporated in the loss to train a CNN model with point supervision (Bearman et al., 2016). Priors of shallow image features were employed in the loss function (Lin et al., 2016; Tang et al., 2018) to propagate information from scribbles to unmarked pixels.

### 6.3 Weakly-Supervised Segmentation in Medical Image

In the scenario of medical images, as full annotation is expensive due to professional clinical input being required and time involved, and priors about objects are usually well-established, interest in weakly-supervised segmentation is increasing rapidly.

DeepCut (Rajchl et al., 2016) adopted an iterative updating procedure to train a CNN model for fetal MRI segmentation based on a bounding box.

Other prior work (Kervadec et al., 2019) introduced a differentiable penalty in the loss function to enforce inequality constraints, which was applied to the cardiac, vertebral body, and prostate segmentation on MRI images.

Kervadec et al. (Kervadec et al., 2020) leveraged the tightness prior via constrained loss for the segmentation of spinal and brain MRI. Edge information was utilised in PseudoEdgeNet (Yoo et al., 2019), which trained the model to segment the nuclei with point annotations.

Prior work (Qu et al., 2020) generated two types of coarse labels from point annotations to train a model for the segmentation of histopathology images.

In another study of the prior art, (Valvano et al., 2021), the model was trained with an adversarial game for segmentation from scribble annotations in MRI images.

The MRI-SegFlow (Kuang et al., 2020) also adopted the idea of automatic annotation and proposed a two-stage process for VB segmentation. It adopted a rule-based method to automatically generate the suboptimal region of interest (ROI) and trained the CNN model with the suboptimal ROI.

However, by stark contrast the process as provided by the present invention, the suboptimal ROI in MRI-SegFlow was not further optimized with the CNN training process.

Furthermore and disadvantageously as noted by the present inventor, the rule-based method of MRI-SegFlow required further modification to transfer to other tissues.

### 7. METHODOLOGY AND PROCESSES OF THE PRESENT INVENTION

Again, although the present invention is applicable to numerous types of body tissues, and multiple body tissues for some regions of interest within a subject, the process and examples provided as follows are in respect of spinal analysis for ease of specific reference and demonstration of the present invention, and such an exemplary embodiment in view of being directed towards spinal analysis has been termed “Spine-GFlow”.

### 7.1 Overview of Present Invention (Spine-GFlow)

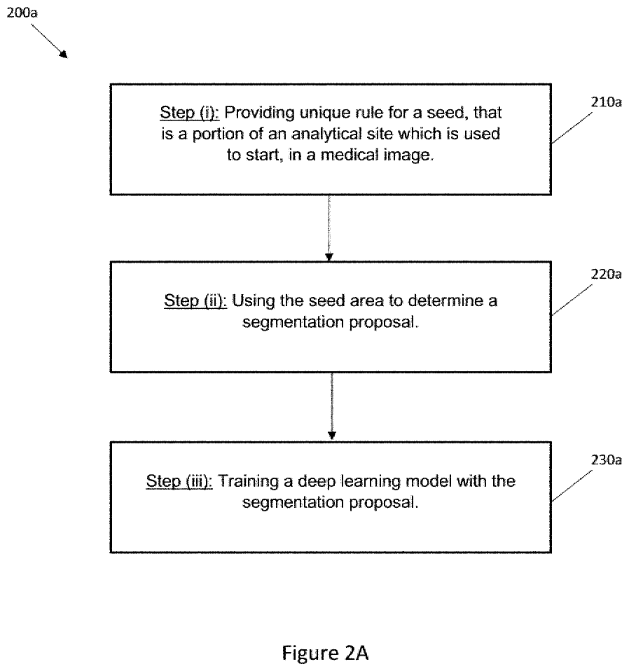

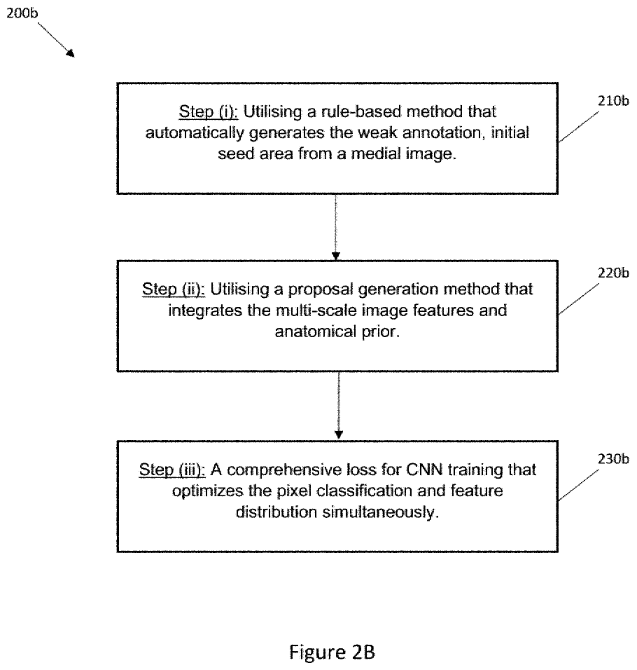

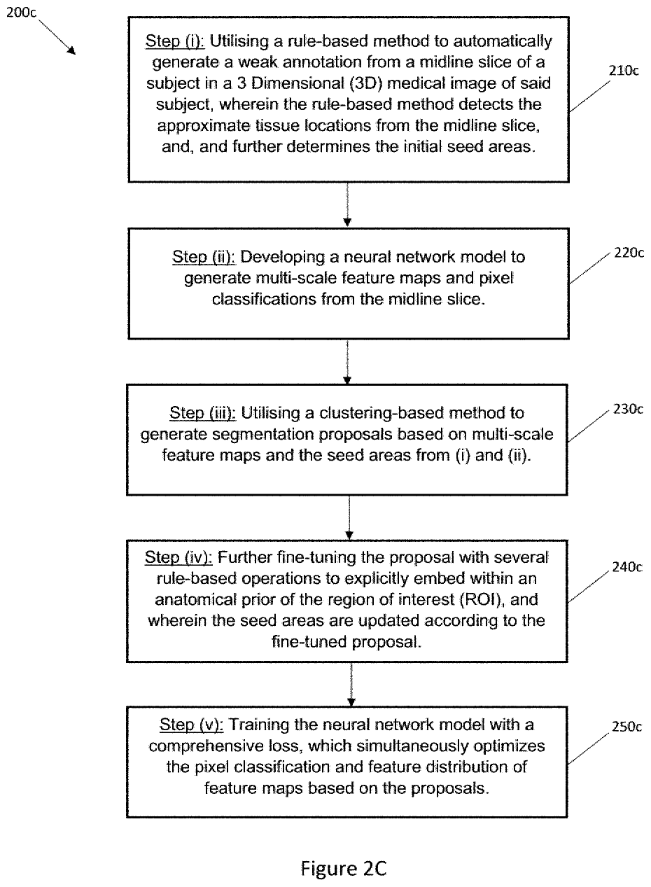

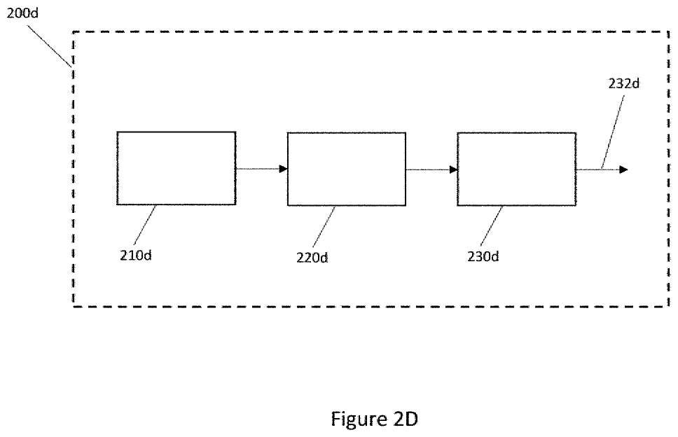

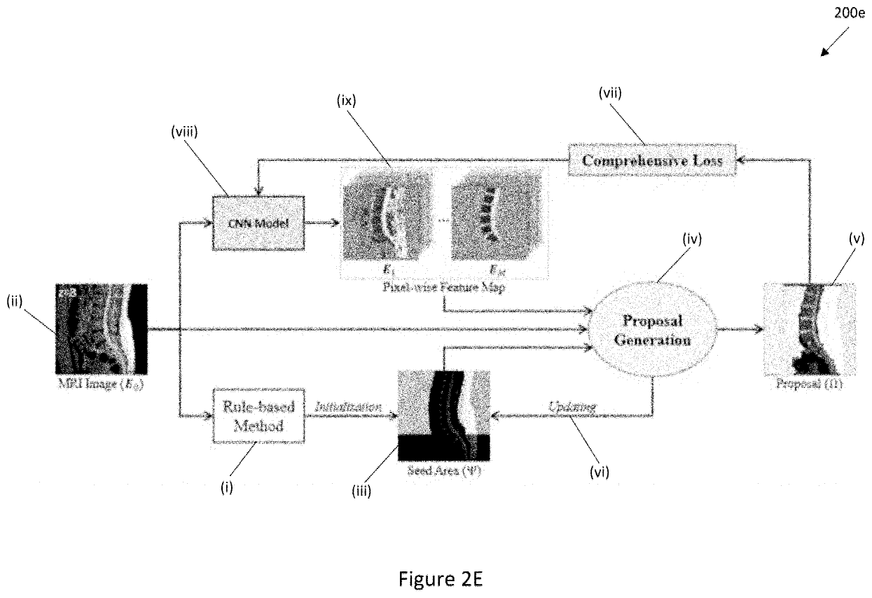

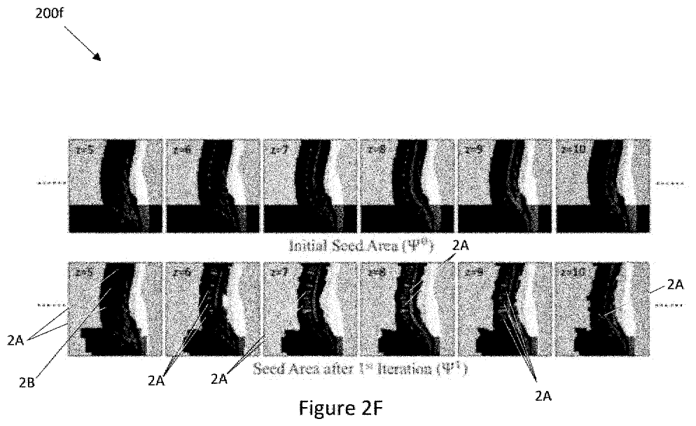

As is shown in FIG. 2A there is an exemplary embodiment of a process 200a of the present invention.

The process 200a provides a three-dimensional (3D) tissue model of one or more tissues or tissue types of a subject,

The process 200a includes the steps of:
(i) 210a providing unique rule for a seed, that is a portion of an analytical site which is used to start, in a medical image;
(ii) 220a using the seed area to determine a segmentation proposal; and
(iii) 230a training a deep learning model with the segmentation proposal.

As is shown in FIG. 2B there is a further exemplary embodiment of a process 200b of the present invention for tissue modelling from a medical image of a subject, for forming a three-dimensional.

The process 200b includes the steps of:
(i) 210b utilising a rule-based method that automatically generates the weak annotation, initial seed area from a medial image;
(ii) 220b utilising a proposal generation method that integrates the multi-scale image features and anatomical prior; and
(iii) 230b a comprehensive loss for CNN training that optimizes the pixel classification and feature distribution simultaneously.

As is shown in FIG. 2C there is a further exemplary embodiment of a process 200c of the present invention for tissue modelling of a subject of one or more tissue type at a region of interest (ROI) of said subject, for forming a three-dimensional (3D) model of a region of interest (ROI) of a subject with one or more tissue types.

The process 200c includes the steps of:
(i) 210c utilising a rule-based method to automatically generate a weak annotation from a midline slice of a subject in a 3 Dimensional (3D) medical image of said subject, wherein the rule-based method detects the approximate tissue locations from the midline slice, and, and further determines the initial seed areas;
(ii) 220c developing a neural network model to generate multi-scale feature maps and pixel classifications from the midline slice;
(iii) 230c utilising a clustering-based method to generate segmentation proposals based on multi-scale feature maps and the seed areas from (i) and (ii);
(iv) 240c further fine-tuning the proposal with several rule-based operations to explicitly embed within an anatomical prior of the region of interest (ROI), and wherein the seed areas are updated according to the fine-tuned proposal; and
(v) 250c training the neural network model with a comprehensive loss, which simultaneously optimizes the pixel classification and feature distribution of feature maps based on the proposals.

As is shown in FIG. 2D there is an exemplary embodiment of a system (200d) of the present invention for tissue modelling of a subject of one or more tissue type at a region of interest (ROI) of said subject, for forming a three-dimensional (3D) model of a region of interest (ROI) of a subject with one or more tissue types.

The system includes:
an input module 210d for receiving a 3 Dimensional (3D) medical image containing a plurality of 2 Dimensional (2D) slices of a subject of a region of interest (ROI) of said subject;
a processor and a neural network 220d for providing the process of:
(i) using a rule-based method to automatically generate a weak annotation from the plurality of 2 Dimensional (2D) slices of said subject, wherein the rule-based method detects the approximate tissue locations from the plurality of 2 Dimensional (2D) slices, and further determines the initial seed areas;
(ii) developing a neural network model to generate multi-scale feature maps and pixel classifications from the plurality of 2 Dimensional (2D) slices;
(iii) utilising a clustering-based method to generate segmentation proposals based on multi-scale feature maps and the seed areas;
(iv) further fine-tuning the proposal with several rule-based operations to explicitly embed within an anatomical prior of the region of interest (ROI), and wherein the seed areas are updated according to the fine-tuned proposal; and
(i) training the neural network model with a comprehensive loss, which simultaneously optimizes the pixel classification and feature distribution of feature maps based on the proposals; and
   an output module 230d for providing an output representation a three-dimensional (3D) model of a region of interest (ROI) of the subject 232d with one or more tissue types.

As is shown in FIG. 2E there is an exemplary embodiment of a process (200e) of the present invention, exemplified by an embodiment directed towards spinal analysis for a regions of interest (ROI).

In accordance with the present invention, seed areas are first initialized with a rule-based method.

In the iterative optimization procedure, the proposals are generated based on the acquired MRI image (or other medical image), pixel-wise feature maps, and seed areas.

The seed areas are updated for the next iteration.

The generated proposals are further used to calculate a comprehensive loss to train a convolutional neural network (CNN) model.

An example (200e) of the initial seed areas and seed areas after 1st iteration is shown in FIG. 2F (2A: VB, 2B: IVD, 2C SC, 2D: background), which shows that the initial seed areas are only in a few slices that are not necessarily the same.

The seed areas expand to adjacent slices and get closer to the proposals during the updating process.

The overall process of the embodiment of the present invention is depicted in FIG. 2A.

A rule-based method (i) is first applied on the MRI image E₀, in this embodiment of the spine, which utilises anatomical priors of tissue including texture, relative location, and size, to detect the approximate tissue locations and a rough spinal region.

For each tissue, in the present embodiment, its locations are only detected in its midline sagittal slices.

The detection result is utilised to (iii) initialise the seed areas Ψ, and the initial seed areas are served as the automatic weak annotation of the framework of the invention.

For each tissue type, the initial seed area consists of small 3D neighborhoods around the tissue locations, which are not necessarily in the same slice due to potential scoliosis or other distortions or inconsistencies in other ROIs and tissue, for example as is shown FIG. 2B).

The initial seed area of the background is determined according to the rough spinal region. More details about the rule-based seed area initialisation will be discussed in Section 7.2

Further, the MRI image E₀ is fed into a CNN model that can generate multiple pixel-wise feature maps, E₁, . . . , E_{M}, with different scales.

The proposal generation method (iv) integrates the MRI image E₀ (ii), multi-scale feature maps, E₁, . . . , E_{M}, and seed areas Ψ (iii) to generate the segmentation proposals Ω (v).

Each proposal consists of pixels belonging to a specific tissue or background, and pixels that are not in any proposals are defined as ambiguous pixels.

The seed areas are also updated (vi) for the next iteration of proposal generation (iv), which expand to adjacent slices and get closer to the proposals during the updating as is shown progressively though FIG. 2B.

More details about proposal generation will be discussed in Section 7.3.

Based on the proposals, a comprehensive loss (vii) is calculated to train the CNN model (viii), which will be discussed in detail in Section 7.3.

In accordance with the present invention, only pixels within the proposals are involved in CNN training, and ambiguous pixels are ignored from processing and analysis.

The proposal generation (iv) and CNN training (viii) are conducted iteratively.

In each iteration, the MRI image (ii) is first fed into the CNN mode (viii), which will produce multi-scale feature maps (ix).

Then, based on the feature maps, the proposals are generated (iv), which are further used to calculate the comprehensive loss for CNN training (vii).

The optimized CNN model can produce the feature maps (ix) for better proposal generation in turn.

In the following statement:
(u, v) represents the 2D coordinates of a pixel in a 2D image, and
p=(x, y, z) represents the 3D coordinates of a pixel in a 3D scan,
where z is the index of the slice.
Let t=0,1,2,3 represent the background, VB, IVD, and SC.

### 7.2 Rule-Based Initialisation—Spinal Example

In the embodiment of the present invention, a rule-based method is utilised to generate the initial seed areas for VB, IVD, SC, and background.

The VB area is identified first via gradient thresholding, size selection, and location selection, which determines approximate VB locations as well as a rough spinal region.

Then, the IVD and SC are localized based on their relative location to the VB.

Further, the seed areas are initialised according to the tissue locations and rough spinal region.

In gradient thresholding, the normalized image gradient gₙ and amplified image gradient gₐ are defined as:

$${g}_{n} ( u , v ) = g ( u , v ) / ave ( u , v ) \qquad ( 1 )$$

$${g}_{a} ( u , v ) = g ( u , v ) * ave ( u , v ) \qquad ( 2 )$$

where g(u, v) is the image gradient magnitude calculated with a Sobel operator, and ave(u, v) is the average pixel value in the 3×3 neighborhood at (u, v).

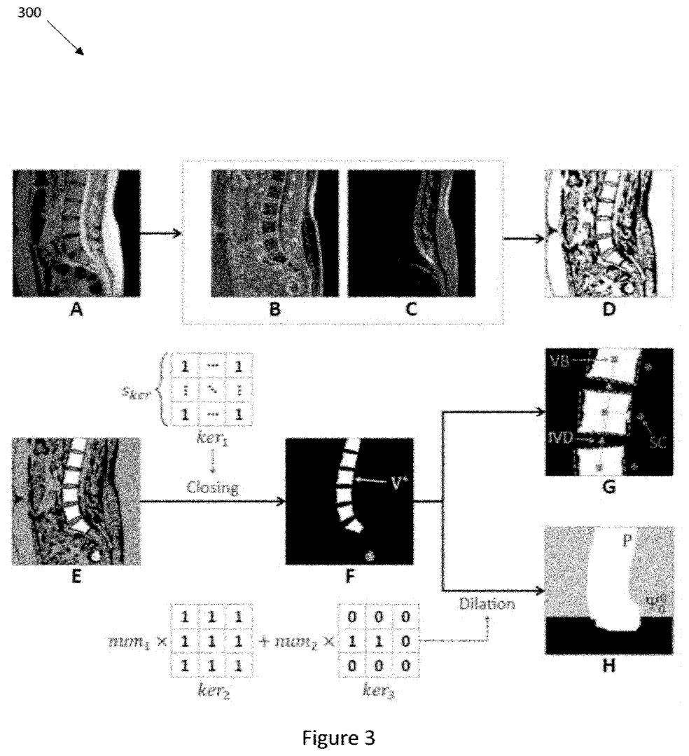

FIG. 3A-3H shows a rule-based seed area initialization (300). Considering the whole MRI scan of FIG. 3A as a 3D volume, the gₙ and gₐ are calculated in transverse, coronal, and sagittal views separately.

The normalized and amplified gradients of 3D volume, Gₙ and Gₐ, are the pixel-wise maximum of gₙ and gₐ in three views, respectively FIG. 3b and FIG. 3c).

The potential VB area is defined as: V={p: Gₙ(p)<Tₙ, Gₐ(p)<Tₐ}, where Tₙ and Tₐ are two threshold values as shown in FIG. 3d.

The potential VB area is further processed via size selection and location selection as is shown in FIG. 3E and FIG. 3F.

The potential VB area is first considered as several 2D Connected Components (CCs) in each slice and find the Minimum Bounding Rectangle (MBR) for each 2D CC.

The height, width, and aspect ratio of each MBR are measured and CCs removed whose measurements are out of a certain range.

Then, the processed VB area is treated as several three dimensional (3D) CCs in an MRI scan.

For each 3D CC, the thickness is measured (i.e., how many slices it spans) and select the 3D CC with the required thickness.

The midline slice of each selected 3D CC is projected onto one image and morphological closing using a square kernel ker₁ with a size of sₖₑᵣ is applied.

The morphological closing can merge VB projections and isolate non-VB projections. All 3D CCs corresponding to isolated projections are removed, and the remaining CCs are denoted as V*. The tissue locations lₜ are determined based on V*.

The MBR is found for the midline slice of each 3D CC in V*.

For each VB, the center location l₁=(x₁, y₁, z₁) and width w₁ are measured from the MBR.

Further, the center locations of IVD l₂ and SC l₃ are determined by:

$${l}_{2}^{n} = ( ( {x}_{1}^{n} + {x}_{1}^{n + 1} ) / 2 , ( {y}_{1}^{n} + {y}_{1}^{n + 1} ) / 2 , ( {z}_{1}^{n} + {z}_{1}^{n + 1} ) / 2 ) \qquad ( 3 )$$

$${l}_{3}^{n} = ( {x}_{1}^{n} - α * sin {θ}^{n} * {w}_{1}^{n} , {y}_{1}^{n} + α * cos {θ}^{n} * {w}_{1}^{n} , {z}_{1}^{n} ) \qquad ( 4 )$$

Where:

θⁿ=arctan((y₁ⁿ⁺¹−y₁ⁿ⁺¹)/(x₁ⁿ⁺¹−x₁ⁿ⁻¹)),
α is a constant which is set to 0.80, and (x₁ⁿ, y₁ⁿ, z₁ⁿ) and (x₁^{n±1}, y₁^{n±1}, z₁^{n±1}) are the center locations of three adjacent VBs (FIG. 3G).

The initial seed areas Ψₜ⁰ of VB, IVD, and SC are defined as 3D neighborhoods at corresponding center locations.

To determine the initial seed area of background Ψ₀⁰, the midline slice of each 3D CC is projected in V* onto one image and apply the morphological dilation to generate a rough spinal region P.

The morphological dilation takes num₁ and num₂ iterations with the kernel ker₂ and ker₃, respectively.

Let the location of the lowest VB in V* be l₁^{max}=(x₁^{max}, y₁^{max}, z₁^{max}) while Ψ₀⁰ is defined as: Ψ₀⁰={p|(x, y)∉P, x<x₁^{max}} and is the same in each MRI slice as is shown in FIG. 3H).

The specific configuration of rule-based initialisation is described in Section 8.2.

FIG. 3A-3H shows the rule-based seed area initialisation.

FIG. 3A presents an MRI, whose Gₙ and Gₐ are presented in FIG. 3B and FIG. 3C.

FIG. 3D is the potential VB area V.

FIG. 3E and FIG. 3F illustrate the size and location selection on V, and the white area in Figure E and Figure F represents the selection result.

FIG. 3G presents center locations of tissues.

FIG. 3H shows the projection of rough spine area P and the initial seed area of the background Ψ₀⁰. ker₁, ker₂, and ker₃ are 3 kernels for location selection and determination of the rough spinal region.

### 7.3 Proposal Generation

Unlike most iterative optimization methods, which generate proposals based on CNN output, the process of the present invention combines different levels of information by integrating multi-scale feature maps in proposal generation.

In the present embodiment of the invention. a clustering-based method is applied on each feature map first to divide pixels into several clusters, and each pixel cluster is further decomposed into several CCs.

Specific CCs are selected according to each seed area and assembled into the corresponding proposal, which is further fine-tuned with several rule-based operations to explicitly embed the anatomical prior.

Finally, the seed areas are updated based on the proposals and pixel clustering results for the next iteration of proposal generation.

### 7.3.1 Clustering-Based Pixel Division

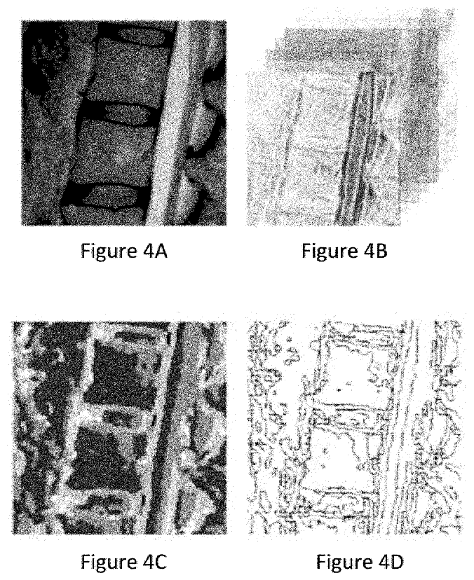

FIG. 4A and Figure B present an MRI patch and one of its feature maps generated by the CNN model, FIG. 4C is the clustering result of the feature map, and FIG. 4D is the pixel division based on the clustering result.

In this example, the k-means algorithm is utilised for pixel clustering, which iteratively conducts the assignment and update steps. In the assignment step, each pixel is assigned to the cluster with the most similar mean feature.

The assignment step produces a set of pixel clusters C as shown in FIG. 4C, which is defined as:

$${C}_{k} = \{ {p}_{i} : {‖ {e}_{i} - {ρ}_{k} ‖}_{2} \leq {‖ {e}_{i} - {ρ}_{j} ‖}_{2} ∀ j , 1 \leq j \leq K \} \qquad ( 5 )$$

Where:
   eᵢ is the feature of pixel pᵢ, ρₖ is the mean feature of Cₖ, and
   K is the number of total pixel clusters.

In the update step, the mean feature of each pixel cluster is calculated as:

$${ρ}_{k} = \frac{1}{\vert {C}_{k} \vert} {\sum }_{{p}_{i} ∈ {C}_{k}} {e}_{i} \qquad ( 6 )$$

where |Cₖ| is the number of pixels in Cₖ.

The mean feature is initialised with K randomly selected pixel features from the feature map, and the clustering stops after 10 iterations.

Formally the pixel clustering process is defined on feature map E as:

$$Clu ( E ) = \{ {C}_{k} \} \qquad ( 7 )$$

Pixel clustering is conducted on the original MRI (or other medial image in other or alternate embodiments) E₀ of FIG. 4A and the multi-scale feature maps, E₁, . . . , E_{M}, of FIG. 4B, generated by the CNN model, individually.

For E₀={vᵢ}, vᵢ represents the pixel value of pᵢ, which is the feature with the smallest scale.

For Eₘ={eᵢ}ₘ (m≥1) the feature of each pixel is normalized as \tilde{e}ᵢ=eᵢ/∥eᵢ∥ before clustering.

Since the location of each pixel is not involved in the clustering process, each pixel cluster may not be spatially aggregated, which can be represented as Cₖ=Uₙ cc_{k,n}, where cc_{k,n} is the 2D CC in Cₖ.

Further, pixels in the MRI scan can be divided into multiple 2D CCs (FIG. 4 D) based on the clustering result of E. Formally, we define the pixel division process as:

Div(E)={cc_{k,n}}

### 7.3.2 Pixel Selection

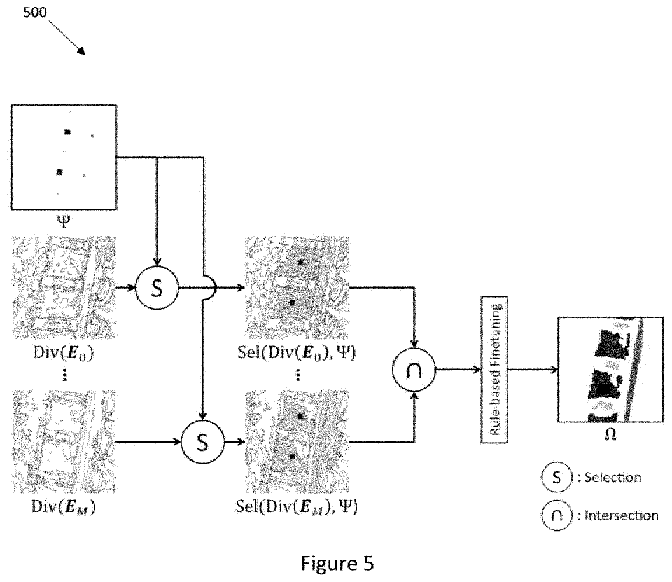

FIG. 5 shows the pixel selection process (500). The 2D CCs in the pixel division result of each feature map are selected according to the seed areas W, as is shown in FIG. 5.

The CC that overlaps with Ψₜ. are select and assembled.

The selection process is defined as:

$$Sel ( \{ {cc}_{kn} \} , {Ψ}_{t} ) = {⋃}_{{cc}_{k , n} ⋂ {Ψ}_{t} \neq ∅} {cc}_{k , n} \qquad ( 8 )$$

and is conducted on the pixel division result of original MRI E₀ and feature maps, E₁, . . . , E_{M}, individually, and the proposals Ωₜ are defined as the intersections of all selection results:

$${Ω}_{t} = {⋂}_{m = 0}^{M} Sel ( Div ( {E}_{m} ) , {Ψ}_{t} ) \qquad ( 9 )$$

The proposals are further fine-tuned with three rule-based operations:
1. 3D Morphological Closing: Considering the tissue should be solid in an MRI scan, 3D morphological closing on the proposals is applied to remove any potential small inner cavities.
2. 3D Morphological Opening: Since the tissue has relatively fixed positions and no drastic shape variation in adjacent slices, 3D morphological opening are applied on the proposals to remove structures with insufficient thickness.
3. Exclusivity: Each pixel can only be in one proposal. If a pixel belongs to more than one proposal, it will be removed from all proposals.

The 2D CCs that overlap with the seed areas are selected and assembled. The intersections of all selection results derived from E₀, . . . , E_{M} are further processed with the rule-based fine-tuning to generate the final proposals To update the seed area of each tissue, the dominant pixel cluster D for each proposal based on the clustering results of feature maps is first determined.

The pixel cluster is selected from the clustering result of each feature map that contains the most pixels in the proposal.

The dominant pixel cluster is the intersection of all selected pixel clusters, which is defined as:

$${D}_{t} = {⋂}_{m = 1}^{M} {arg max}_{{C}_{k} ∈ Clu ( {E}_{m} )} \vert {C}_{k} ⋂ {Ω}_{t} \vert \qquad ( 10 )$$

Note that only the feature map generated by CNN model is utilised, and the original image E₀ is not involved in the determination of dominant pixel cluster. In each round of updating, the seed area will expand to adjacent slices. Let pixel p=(x, y, z) and its slice neighborhood snₚ be defined as snₚ={(x, y, z±1)}.

Let the expanded part of seed area Ψₜ* be defined as Ψₜ*={p: p∈Dₜ, p∉Ωₜ, snₚ∩Ωₜ≠Ø}.

The expanded part covers the pixels from the dominant pixel cluster whose slice neighborhood overlaps with the proposal.

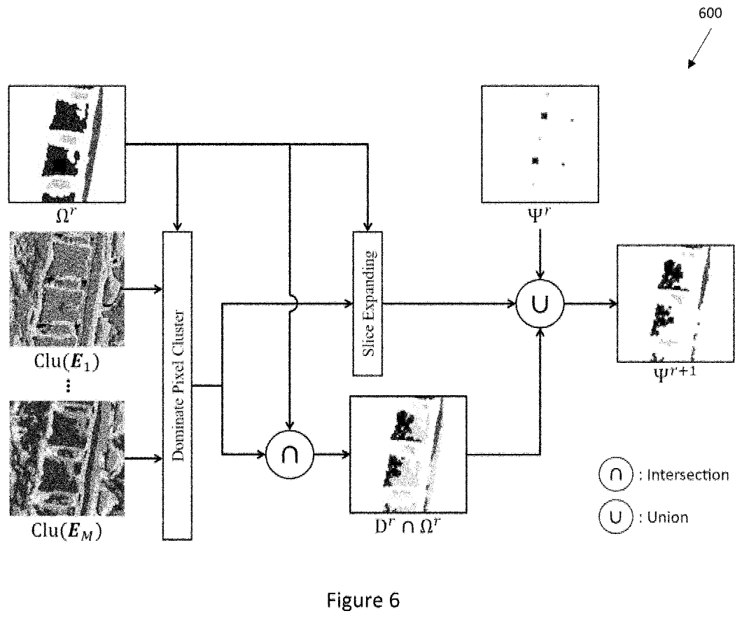

FIG. 6 shows a seed area updating (600) The seed area is updated as is shown in FIG. 6 as follows:

$${Ψ}_{t}^{r + 1} = ( {D}_{t}^{r} ⋂ {Ω}_{t}^{r} ) ⋃ {Ψ}_{t}^{* r} ⋃ {Ψ}_{t}^{r} \qquad ( 11 )$$

where r represents the number of iterations.

For the background, the seed area is simply updated as:

$${Ψ}_{0}^{r + 1} = {Ω}_{0}^{r} \qquad ( 12 )$$

FIG. 6 shows the seed area updating. The dominant pixel cluster is first determined based on the clustering results of feature maps, Clu(E₁), . . . , Clu(E_{M}), and proposal Ω^{r}.

The slice expanded part is further determined.

The updated seed area is the union of previous seed area, slice expanded part, and intersection of dominant pixel cluster and proposal.

### 7.4 Convolutional Neural Network (CNN) Model and Training

### 7.4.1 CNN Architecture

The CNN model in the present embodiment utilises the U-Net++ as the backbone, which can generate multi-scale pixel-wise feature maps from input MRI images.

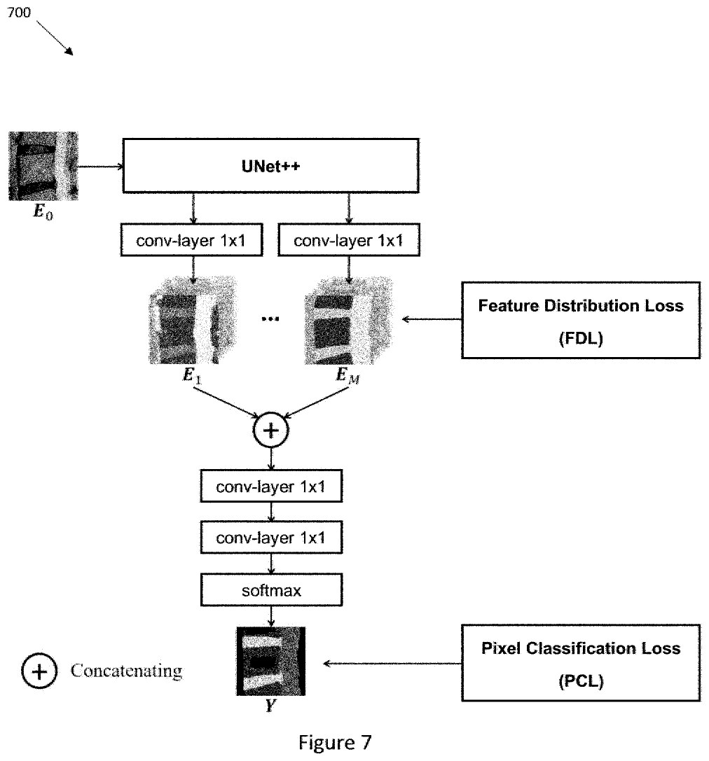

FIG. 7 shows a CNN model and comprehensive loss (700). As illustrated in FIG. 7, the CNN model can generate M feature maps, E₁, . . . , E_{M}, where M is determined by the number of levels in the U-Net++.

In the present embodiment, all feature maps are concatenated and further processed by two convolutional layers (conv-layers) with a kernel size of 1×1 and a softmax layer, which produce the pixel classification Y.

FIG. 7 shows the CNN model and comprehensive loss.

The CNN model of the present embodiment of the invention utilises adopts the U-Net++(Zhou et al., 2019) as the backbone to generate multi-scale feature maps, which are further concatenated and processed by two convolutional layers and a softmax layer to generate pixel classification.

The comprehensive loss consists of the Pixel Classification Loss (PCL) and Feature Distribution Loss (FDL), which optimize the pixel classification and feature distribution of feature maps, respectively.

### 7.4.2 Comprehensive Loss

A comprehensive loss is calculated based on the proposals and consists of the Pixel Classification Loss (PCL) and Feature Distribution Loss (FDL) as is shown in FIG. 7.

The PCL introduces the penalization in the pixel classification Y={yᵢ} generated by the CNN model. For proposal Ωₜ, the PCL is defined as:

$$PCL ( {Ω}_{t} ) = \frac{1}{\vert {Ω}_{t} \vert} {\sum }_{{p}_{i} ∈ {Ω}_{t}} ce ( {y}_{i} , {\hat{y}}_{i} ) \qquad ( 13 )$$

where yᵢ and ŷᵢ are the CNN classification and ground truth of pᵢ and ce( ) is the cross entropy.

In order to optimize the pixel feature distribution of the feature maps, beyond the conventional cross entropy PCL, the FDL is introduced, which encourages the CNN model to generate homogeneous features for pixels from the same proposal, and inhomogeneous features for pixels from different proposals.

For the feature map Eₘ={eᵢ}ₘ (m≥1), the FDL of tissue proposal Ωₜ (t∈[1,3]) is defined as:

$$FDL ( {E}_{m} , {Ω}_{t} ) = - \frac{1}{\vert {Ω}_{t} \vert} {\sum }_{{p}_{i} ∈ {Ω}_{t}} log \frac{exp ( {φ}_{t}^{T} {\tilde{e}}_{i} )}{{\sum }_{s ∈ [ 1 , 3 ] , s \neq t} exp ( {φ}_{s}^{T} {\tilde{e}}_{i} )} \qquad ( 14 )$$

where \tilde{e}ᵢ=eᵢ/∥eᵢ∥ is the normalized pixel feature and φₜ=(Σₚ_{ᵢ}_{∈Ω}_{ₜ}\tilde{e}ᵢ)∥Σₚ_{ᵢ}_{∈Ω}_{ₜ}\tilde{e}ᵢ∥ is the mean feature of Ωₜ.

The numerator encourages each pixel feature to be close to the mean feature of its own proposal, and the denominator pushes each pixel feature away from the mean feature of other proposals.

The FDL of background proposal Ω₀ is defined as:

$$FDL ( {E}_{m} , {Ω}_{0} ) = - \frac{1}{\vert {Ω}_{0} \vert} {\sum }_{{p}_{i} ∈ {Ω}_{0}} log \frac{1}{{\sum }_{s ∈ [ 1 , 3 ]} exp ( {φ}_{s}^{T} {\tilde{e}}_{i} )} \qquad ( 15 )$$

Due to the diversity of pixel features in background, the mean feature for Ω₀ is not calculated and the FDL only encourages the pixel feature to be far away from the mean feature of all tissue proposals.

Only pixels within the proposal are involved in CNN training and ambiguous pixels are ignored.

Furthermore, the average loss is calculated over pixels in each proposal separately, which prevents the weight of the small-size tissue from being diluted.

The final loss is calculated as:

$$Loss = {\sum }_{t = 0}^{3} {\sum }_{m = 1}^{M} {a}_{t , m} \times FDL ( {E}_{m} , {Ω}_{t} ) + {\sum }_{t = 0}^{3} {b}_{t} \times PCL ( {Ω}_{t} ) \qquad ( 16 )$$

where a_{t,m} and bₜ are the weights for different losses.

### 7.4.3 Training Protocol

The CNN model is trained with small image patches instead of the whole MRI to make the model focus on the area covering the tissue proposals.

The patches are randomly selected from the MRI slices, where the proposals of all tissues appear. Overlapping or repetition of selected patches is acceptable.

During proposal generation, to provide the feature map of the whole MRI scan, patches with a constant stride from the input MRI are uniformly selected and merge the feature map of each patch generated by the CNN model.

In the present invention, the CNN model can be trained with different protocols.

First, when a set of unlabelled MRI scans are available, the CNN model can be trained with patches selected from different MRI scans. The FDL enforces the model to extract similar features for pixels of the same tissue in different MRI scans, which helps the model learn general features.

After each iteration of training, proposals of all MRI scans are updated simultaneously based on the trained model.

This training protocol may be called holistic training.

The unlabelled MRI scans can be simply collected as a clinical routine.

Since no manual annotation is required in the present invention, in contrast to systems and processes and systems of the prior art, the framework of the invention can provide another CNN training protocol called individual training, where the CNN model is trained on the target MRI directly.

In individual training, patches are selected from the target MRI only, which makes the model adapt to potential feature variations in each MRI scan and advantageously allows the present invention to boot up with only one MRI scan.

To obtain better performance, the present invention can take advantage of both holistic and individual training.

The CNN model is first trained with a set of prepared MRI scans and further fine-tuned on the target MRI. Much fewer patches are used in the fine-tuning process compared with only individual training.

### 8. DATASET AND IMPLEMENTATION DETAILS

### 8.1 Dataset

### 8.1.1 Hong Kong Disc Degeneration Cohort (HKDDC)

The expert anatomically annotated Hong Kong Disc Degeneration Cohort (HKDDC) dataset (Samartzis et al., 2012) included 40 T2-weighted MRI scans collected from 40 different subjects.

This was a population-based dataset with subject recruitment from open advertisement.

The MRI scans were obtained via 3 different MRI machines with resolutions from 448×448 to 512×512.

The detailed composition of MRI scans in the dataset is presented in Table I.

Each MRI scan contained at least 5 lumbar vertebrae from L1 to L5, and there are at least 7 slices in each scan containing annotated spinal structures.

For each scan, the pixel-wise manual annotations of VB, IVD, and SC were provided (from L1 to S1).

All annotation work was completed by three readers who are medically trained, with a fourth reader (a spine surgeon with more than 20 years of clinical experience) to compare the outcomes and confirm precision as well as consistency (the pixel-wise agreement of annotation is 98%).

The MRI scans are split into 20:10:10 as the training, validation, and testing set.

TABLE I
Composition of MRI Scans in the Dataset

| | | Scan | Image | | |
|---|---|---|---|---|---|
| Institution | MRI Machine | Number | Number | Gender | Age |
| Hong Kong | GE Healthcare Signa | 10 | 110 | 60% F | 47.3 ± |
| Sanatorium | | | | | 7.4 |
| Hospital | Siemens Trio | 12 | 180 | 50% F | 50.4 ± |
| | | | | | 8.8 |
| St. Teresa's | Siemens Prisma | 18 | 306 | 50% F | 52.7 ± |
| Hospital | | | | | 8.3 |

### 8.1.2 IVDM3Seg

The MICCAI 2018 Challenge on Intervertebral Disc Localization and Segmentation (IVDM3Seg) dataset contains 16 MRI cases collected from 8 subjects in two stages.

Each case consists of four aligned high-resolution 3D MRI scans with different modalities, including in-phase, opposed-phase, fat, and water, as well as the manually labelled binary mask for IVD.

The MRI was scanned with a 1.5-Tesla MRI scanner of Siemens using Dixon protocol.

Each MRI scan has a size of 256×256×36. More detailed information about the IVDM3Seg dataset could be found on the official website (https://ivdm3seg.weebly.com). For each MRI scan, the focus was only on the area lower than the T11 vertebra (lumbar region).

### 8.2 Implementation Details

### 8.2.1 Rule-Based Initialisation Configuration

The rule-based initialisation was experimentally configured according to the training set. For the HKDDC dataset, in the gradient threshold, the threshold values for the normalized and amplified image gradients Tₙ and Tₐ were set as 2.5 and 0.2.

In size selection, the minimum (min) and maximum (max) we calculated for dimensions of 10 VBs randomly selected from the training set and determined the requested range as [0.7×min, 1.3×max].

Thus, the requested range for height, width, aspect ratio, and thickness were [20,70], [20,70], [0.5,2], and [5,15], respectively.

In the morphological closing of location selection, the kernel size wₖₑᵣ was 25. The sizes of 3D neighborhoods in initial seed areas of VB, IVD, and SC were set as 7×7×3, 3×3×3, and 3×3×1.

The iteration numbers of the morphological dilation for the rough spinal region, num₁ and num₂, were set as 35 and 25.

For the IVDM3Seg dataset, the image intensity and size were significantly different from the clinical T2-weighted MRI of the HKDDC dataset, thus minor adjustments were required in the gradient threshold and size selection.

The normalized image gradient and the amplified image gradient were calculated on fat modality and on opposed-phase modality. The threshold values Tₙ and Tₐ were set as 4.0 and 0.1.

In size selection, the requested range for height, width, and thickness were set as [10,50], [15,50], and [10,30]. The iteration numbers num₁ and num₂ were 20 and 5.

### 8.2.2 Proposal Generation Configuration

For the k-means algorithm, the number of pixel clusters, K, was set as 10. The kernel for the 3D morphological closing and opening in proposal fine-tuning was a cuboid with a size of 5×5×2 and 1×1×3. The 3D morphological opening was not applied in the first iteration.

### 8.2.3 CNN Architecture and Training Configuration

A UNet++ with 4 levels was adopted in the CNN model that could generate 3 pixel-wise feature maps with different scales.

All conv-layers in the model had 64 filters except the output layer, which had 4 filters. For the HKDDC dataset, the input of the CNN model was the patch of raw clinical MRI. For the IVDM3Seg dataset, the input of the CNN model was the patch of the concatenation for 4 modalities of MRIs.

The framework with 3 different CNN training protocols were evaluated:
(1) only Holistic Training (HT),
(2) only Individual Training (IT),
(3) both Holistic and Individual Training (HT+IT).

For HT, the CNN model was trained with only the training set and applied on the target MRI directly without any fine-tuning. The training took 15 iterations.

For each iteration, 5,120 patches were selected from each MRI.

For IT, the CNN model was trained from scratch on only the target MRI, which also took 15 iterations, and 5,120 patches were selected from the target MRI for each iteration. For HT+IT, the CNN model was first pretrained on the training set with 15 iterations, and for each iteration, 3,840 patches were selected from each MRI.

The CNN model was further fine-tuned on each target MRI with 8 iterations.

For the first 7 iterations, 512 patches were selected from the target MRI, and for the last iteration, 3,072 patches were selected.

For all training protocols, all weights of the loss were set as 1.

The mini-batch strategy with a batch size of 16 was adopted. Adam was used as the optimizer with an initial learning rate of 0.0006.

### 8.3 Evaluation Metrics

Two different metrics were adopted to quantitatively evaluate the segmentation performance of the present invention:

Intersection over Union (IoU) and the Dice coefficient (Dice), which were calculated as follows:

$$IoU = \frac{TP}{TP + FP + FN} \qquad ( 17 )$$

$$Dice = \frac{2 TP}{2 TP + FP + FN} \qquad ( 18 )$$

where TP, FP and FN denoted the number of true positive, false positive, and false negative pixels in the segmentation results respectively.

The mean IoU and mean Dice were defined as the average IoU and Dice of all tissues.

### 9. RESULTS

### 9.1 Multi-Tissue Segmentation

The quantitative evaluation results of the multi-tissue segmentation performance achieved by “Spine-GFlow”, an implementation of the present invention on the HKDDC dataset were shown in Table II, and Table Ill showed the IVD segmentation result on the IVDM3Seg dataset.

The framework of the present invention was compared with 3 different CNN training protocols, including HT, IT, and HT+IT.

Furthermore, the method of the present invention was compared with the model trained with the constrained losses in (Kervadec et al., 2019), the automatic annotation of MRI-SegFlow (Kuang et al., 2020), and the full supervision.

The constrained losses (Kervadec et al., 2019) trained the model using small regions within the ground-truth mask, which were similar to the initial seed areas used in the framework of the present invention.

Weak annotation was generated for constrained losses according to (Kervadec et al., 2019).

The MRI-SegFlow (Kuang et al., 2020) provided a rule-based method to generate automatic annotation of VB, and modified parameters were modified and transferred it to IVD and SC.

For all training strategies, the CNN model adopted the same network architecture:

TABLE II
Evaluation of Multi-tissue Segmentation Performance on HKDDC Dataset

| | IVD | | VB | | SC | |
|---|---|---|---|---|---|---|
| Method | IoU | Dice | IoU | Dice | IoU | Dice |
| Constrained | 0.745 ± | 0.854 ± | 0.801 ± | 0.889 ± | 0.794 ± | 0.885 ± |
| Losses | 0.036 | 0.026 | 0.036 | 0.022 | 0.035 | 0.022 |
| MRI-SegFlow | 0.806 ± | 0.892 ± | 0.829 ± | 0.907 ± | 0.782 ± | 0.877 ± |
| | 0.036 | 0.025 | 0.021 | 0.012 | 0.034 | 0.022 |
| Spine-GFlow (HT) | 0.830 ± | 0.907 ± | 0.860 ± | 0.925 ± | 0.809 ± | 0.894 ± |
| | 0.035 | 0.021 | 0.029 | 0.017 | 0.045 | 0.029 |
| Spine-GFlow (IT) | 0.846 ± | 0.916 ± | 0.843 ± | 0.914 ± | 0.807 ± | 0.893± |
| | 0.029 | 0.017 | 0.041 | 0.025 | 0.051 | 0.034 |
| Spine-GFlow | 0.847 ± | 0.917 ± | 0.866 ± | 0.928 ± | 0.811 ± | 0.896 ± |
| (HT + IT) | 0.028 | 0.016 | 0.022 | 0.012 | 0.040 | 0.026 |
| Full Supervision | 0.830 ± | 0.907 ± | 0.859 ± | 0.924 ± | 0.846 ± | 0.916 ± |
| | 0.039 | 0.024 | 0.041 | 0.024 | 0.026 | 0.015 |

TABLE III
Evaluation of IVD Segmentation Performance on IVDM3Seg Dataset

| Method | IVD IoU | IVD Dice |
|---|---|---|
| Constrained Losses | 0.773 ± 0.015 | 0.872 ± 0.013 |
| MRI-SegFlow | 0.783 ± 0.021 | 0.878 ± 0.017 |
| Spine-SegLoop (HT) | 0.792 ± 0.017 | 0.884 ± 0.014 |
| Spine-SegLoop (IT) | 0.812 ± 0.018 | 0.895 ± 0.015 |
| Spine-SegLoop (HT + IT) | 0.820 ± 0.015 | 0.901 ± 0.013 |
| Full Supervision | 0.845 ± 0.015 | 0.916 ± 0.013 |

The results showed that the “Spine-GFlow” process of the present invention consistently outperformed the model trained with constrained losses (Kervadec et al., 2019) and MRI-SegFlow (Kuang et al., 2020) for all tissues.

HT and IT achieved similar overall performance. For VB, HT produced 1.7% higher IoU and 1.1% higher Dice than IT on the HKDDC dataset.

For IVD, IT achieved 1.6% higher IoU and 0.9% higher Dice than HT on the HKDDC dataset, as well as 2.0% higher IoU and 1.1% higher Dice on the IVDM3Seg dataset. For SC, there was no significant difference between these two protocols.

By combining HT and IT, the framework of the invention could further improve segmentation accuracy for all tissues. Moreover, the “Spine-GFlow” process of the present invention with HT+IT obtained performance comparable to the model trained with full supervision.

The results from the present invention achieved only 2% lower Dice for SC, and even 1% and 0.4% higher Dice for IVD and VB on the HKDDC dataset.

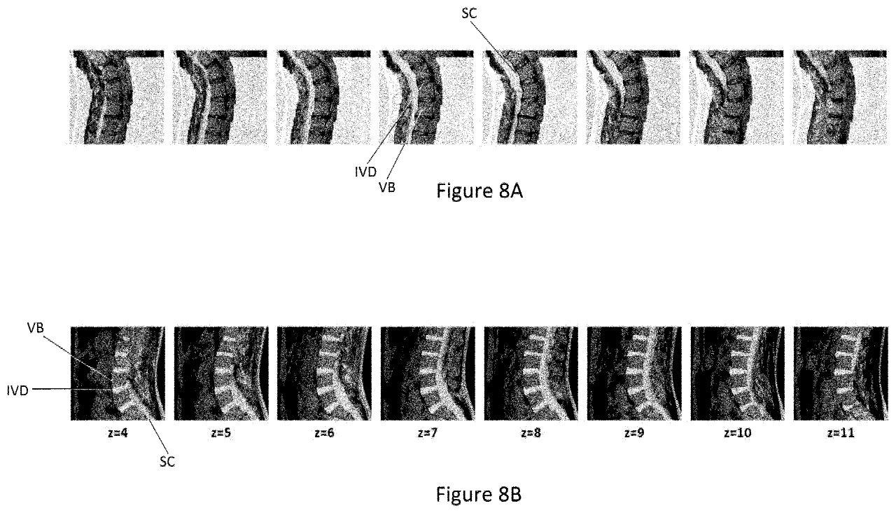

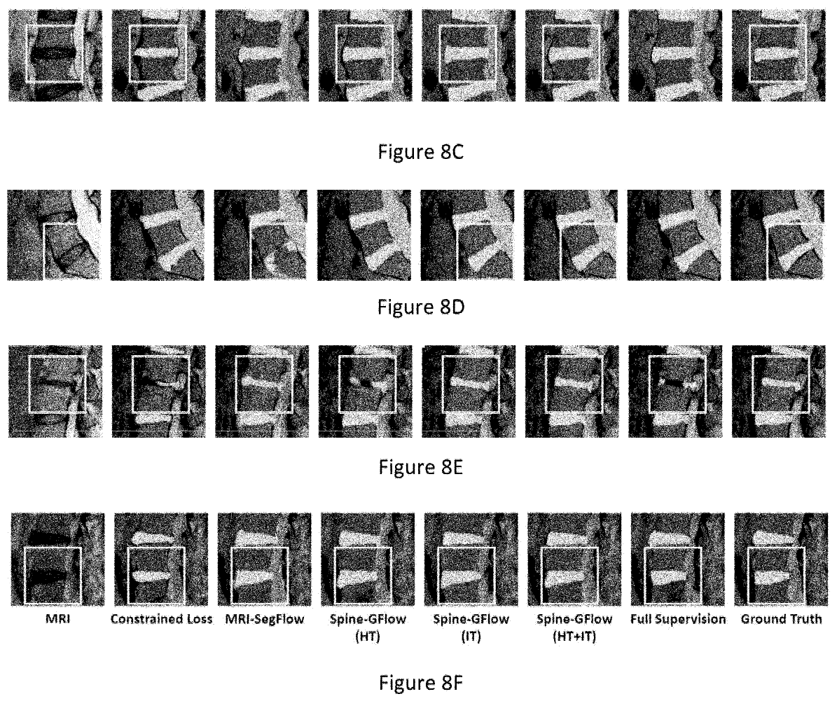

FIG. 8 shows visually presented several multi-tissue segmentation results on the HKDDC dataset.

FIG. 8A and FIG. 8B illustrate the initial seed areas and multi-tissue segmentations, respectively, produced by the process of the present invention on an MRI scan displaying alignment deformity.

The result showed that initial seed areas were presented in different slices since the midline sagittal slices of each tissue were different.

The process of the present invention was shown to be able to adapt the alignment deformity and produce accurate segmentation on different slices.

FIG. 8C to FIG. 8F visually compared the segmentation on MRI patches produced by different methods for regions of interest (ROIs).

The results showed and demonstrated that the process of the present invention could identify the shape detail, such as corners and potential deformity, better than the model trained with constrained losses as shown in FIG. 8C and reduce the noise and shape distortion compared with MRI-SegFlow as is shown in FIG. 8D.

Compared with the framework using only IT, the framework with both HT and IT could reduce noise in the result as shown in FIG. 8D.

For some extreme variations in image features caused by pathologies, such as the Marrow change as shown in FIG. 8E, the process of the present invention with the IT could adapt better than other methods and produce a more accurate result.

Moreover, for the image with low contrast as shown in FIG. 8F, the process of the present invention with the IT also showed high robustness.

FIG. 8A and FIG. 8B present the initial seed areas and multi-tissue segmentation VB, IVD, SC) produced by Spine-GFlow on an MRI scan with alignment deformity. FIG. 8C-FIG. 8F are the visual comparisons of multi-tissue segmentation produced by different methods on MRI patches.

### 9.2 Ablation Study

To further investigate the effect of different components in the framework of the invention, the ablation study was conducted on the HKDDC dataset.

In the process and system of the present invention, the proposals were generated based on multi-scale feature maps produced by the CNN model.

To investigate the effect of integrating the multi-level information, a different proposal generation strategy adopted by (Rajchl et al., 2016) was compared, which produced proposals by applying conditional random field (CRF) on the CNN output.

For a fair comparison, the generated proposals were further fine-tuned with the same rule-based operations above, and the CNN model was trained with the same protocol and loss function. We denoted this variant as Spine-Glow (P−).

The process and system of the present invention introduced the FDL in the CNN training process in addition to the conventional cross entropy PCL to encourage the CNN model to extract more discriminative pixel features.

In order to validate the effect of Feature Distribution Loss (FDL), it was compared it to the framework where the CNN model was trained with only Pixel Classification Loss (PCL). The rest of the framework was kept unchanged and denoted this variant as Spine-Glow (L−).

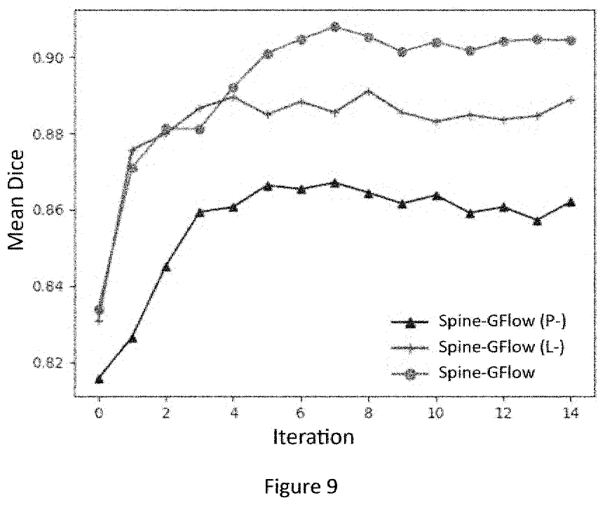

As is shown, FIG. 9 presents the evolution of segmentation performance during the HT process, showing that with multi-scale feature maps and FDL, the CNN model of the standard Spine-GFlow embodiment of the present invention, was trained more efficiently.

Without FDL, the CNN model of Spine-GFlow (L−) achieved the same learning speed as the standard framework at the beginning of HT; however, its performance did not further improve after the 5th iteration, and after HT its mean Dice were 1% lower than the standard Spine-GFlow. In the Spine-GFlow (P−), the proposals generated based on the only model output more significantly reduced the training efficiency of the CNN model, and after HT its mean Dice were 4% lower than the standard framework. Moreover, as presented in Table IV, after HT and IT, the standard Spine-GFlow ultimately obtained better performance than the other 2 variants.

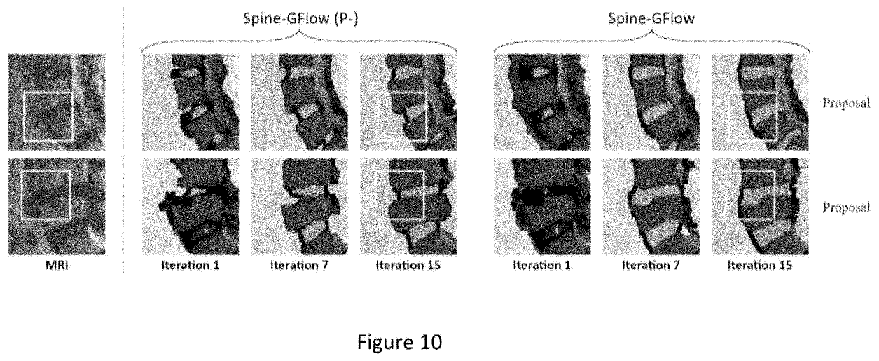

As is shown in FIG. 10, there is presented the proposals generated by Spine-GFlow (P−) and standard Spine-GFlow in different iterations, which showed that the proposals generated with multi-scale feature maps could provide more shape details. Furthermore, when the tissue boundary was not clear, integrating the multi-scale feature maps could avoid the proposals invading the wrong area.

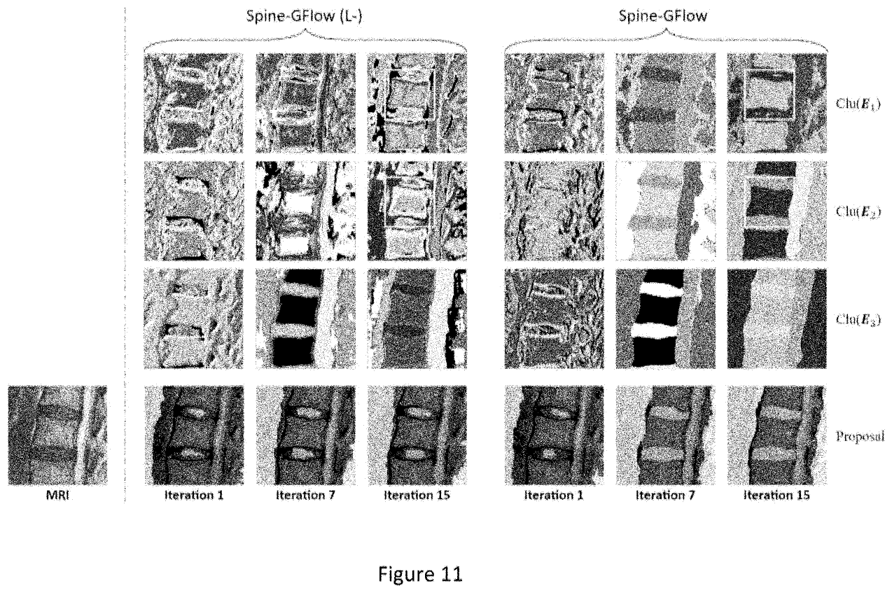

As is shown in FIG. 11, there is presented the pixel clustering result of feature maps generated by the CNN model of Spine-GFlow (L−) and standard Spine-GFlow and the corresponding proposals for regions of interest (ROIs).

Several rule-based fine-tuning operations were adopted in the proposal generation to explicitly embed the anatomical prior, which could effectively reduce the error in the proposals.

It has been demonstrated that the model trained with FDL could generate feature maps whose pixel clustering results could better reflect the true spatial distribution of different tissues with less noise, especially for feature maps with small scales.

More specifically, based on the feature maps of the model trained with FDL, most pixels of the same tissue would be divided into the same pixel cluster, which could help generate more accurate proposals and in turn improve the CNN training process.

TABLE IV
The Ultimate Performance of Three Versions of
Spine-GFlow after HT and IT.

| Framework | mean IoU | mean Dice |
|---|---|---|
| Spine-GFlow (P-) | 0.773 ± 0.053 | 0.870 ± 0.037 |
| Spine-GFlow (L-) | 0.809 ± 0.044 | 0.894 ± 0.029 |
| Spine-GFlow | 0.841 ± 0.026 | 0.914 ± 0.015 |

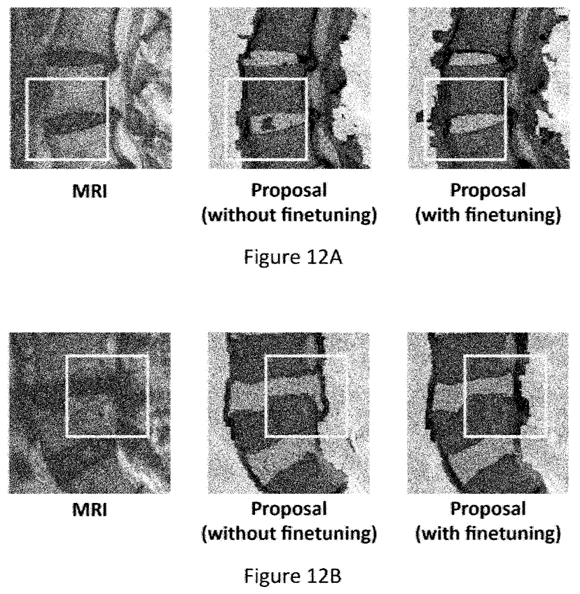

Referring to FIG. 12A and FIG. 12B, there is presented two examples of rule-based fine-tuning that showed fine-tuning could fill the cavity (FIG. 12A) and remove error pixels (FIG. 12B) in the proposals, for regions of interest (ROIs).

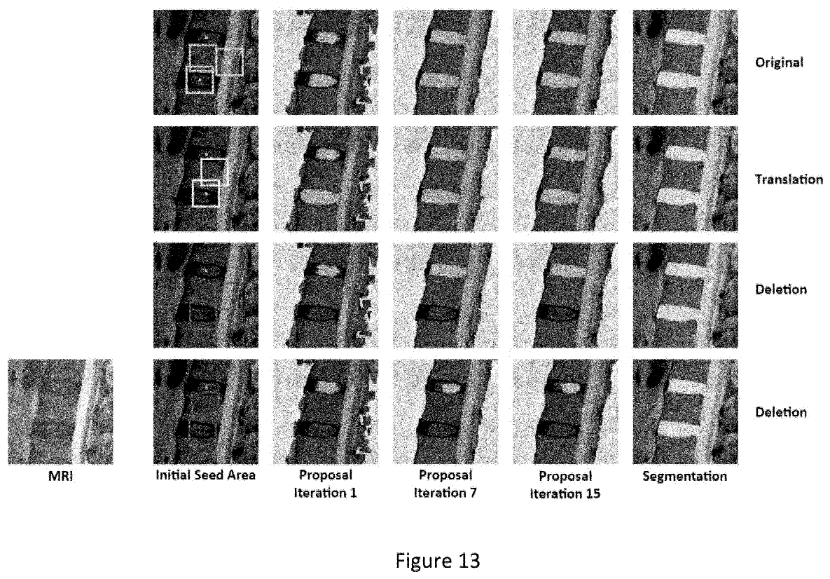

Referring to FIG. 13 there are presented several examples of proposals and segmentation results produced with the defective initial seed areas.

The initial seed areas were manipulated with translation and deletion to simulate potential defects.

The result demonstrated that location deviation did not significantly affect the final proposals and segmentation result. The partial absence led to missing corresponding tissue in the final proposals but had no obvious influence on the segmentation result.

### 10. DISCUSSION OF INVENTION AND RESULTS

### 10.1 Present Invention

The present invention provides a system and process, for robust multi-tissue segmentation of multi-tissue anatomical locations within the body of a human or an animal.

However, the present invention is also applicable to single tissue-type analysis and modelling, although in embodiments is particularly advantageous lumbar spine analysis, and preferably by utilisation of MRI images, although in other embodiments other imaging techniques may be utilised.

Advantageously and in comparison, to systems and processes as proposed by the prior art, the present invention does not require any manual annotations.

In accordance with the present invention:
(i) a rule-based method is first adopted to automatically generate the weak annotation. This rule-based method detects the approximate tissue locations, and in the example of spinal analysis detects the rough spinal region, and further determines the initial seed areas.
   For each tissue, the locations are only detected in a medial image sliced through the body, again in the case of spinal analysis in its midline sagittal MRI slices, thus the initial seed area is not necessarily in the same slice due to potential alignment deformity in spinal applications, which helps the framework adapt to the case with scoliosis for example.
(ii) A CNN model is developed to generate multi-scale feature maps and pixel classifications from the MRI image.
(iii) A clustering-based method is adopted to generate the segmentation proposals based on multi-scale feature maps and the seed areas.
(iv) The proposals are further fine-tuned with several rule-based operations to explicitly embed the anatomical prior, and the seed areas are updated according to the fine-tuned proposals.
(v) Next, the CNN model is trained with a comprehensive loss, which simultaneously optimizes the pixel classification and feature distribution of feature maps based on the proposals. By iteratively conducting the proposal generation and CNN training, we can obtain a CNN model for accurate multi-tissue segmentation. Since no manual annotation is required in the framework of the invention, it can automatically fine-tune the CNN model on the target MRI to improve the robustness of the model against image feature variation.

### 10.2 Comparative Study Results

Within the comparative studies, 2 independent datasets: HKDDC (containing the MRI scans obtained from 3 different machines) and IVDM3Seg were utilised.

The present invention was quantitatively evaluated with three different CNN training protocols, and compared with a CNN model trained with constrained loss (Kervadec et al., 2019), MRI-SegFlow (Kuang et al., 2020), and full supervision. The results showed that the framework consistently outperforms the constrained loss (Kervadec et al., 2019) and MRI-SegFlow (Kuang et al., 2020) for all tissues.

Compared with the constrained loss, the process of the present invention could produce the result with more shape details, which is important for detecting potential deformity.

Compared with the MRI-SegFlow, the present invention can iteratively optimize the proposals for CNN training, the CNN model generates more accurate results with less noise. HT obtains higher segmentation accuracy on VB than IT, while for the IVD, IT performs better.

Since the features of VB such as shape and pixel intensity are more consistent than those of IVD, the model trained with HT performed better given it can learn more general features. Otherwise, for IVD, the model trained with IT can adapt to large individual variations better than with only HT.

By combining HT and IT, the present invention can further improve accuracy on all tissues and achieve a performance comparable with a model trained with full supervision.

IT can also improve the robustness of the framework against the drastic feature variations caused by pathology or low image quality, such as contrast, which helps the method obtain more accurate results than the weakly-supervised and supervised methods in some extreme cases. Furthermore, the multi-source dataset also demonstrated the generalizability of the present invention.

Unlike most iterative optimization methods, which generate proposals based on the CNN output, the framework integrates the multi-scale feature maps generated by the CNN model for proposal generation. The output of a CNN model trained with incomplete annotation usually tends to have smooth contours, and the proposals generated with CNN output will lose shape details, especially for tissues with shape deformities.

Furthermore, since the tissue boundaries are sometimes fuzzy, such as the edge between IVD and the background, refining methods using low level information such as CRF cannot effectively avoid errors, which will significantly reduce the training efficiency of the CNN model and its ultimate performance.

In addition to the conventional cross entropy PCL, the present invention introduces FDL for the training of the CNN model.

Compared with the model trained with only PCL, the model trained with PCL+FDL can generate more discriminative feature maps, where features have large similarities and differences for pixels belonging to the same and different tissues.

For feature maps with small scales, this effect of feature aggregation brought by FDL is more significant, which can help the clustering-based method generate more accurate proposals and in turn improve CNN training.

The anatomical prior was explicitly embedded in the proposal generation by applying several rule-based fine-tuning operations that utilise the 3D geometry information of adjacent slices. The results show that rule-based fine-tuning can significantly improve the accuracy of the proposals by reducing the potential cavities and errors.

The present invention shows high robustness against suboptimal initial seed areas. Since the rule-based method is adopted to locate the tissue in the MRI scan for the initialisation of seed areas, it will sometimes provide suboptimal results.

It is shown by mimicking two kinds of potential defects in initial seed areas, namely the location deviation and partial absence, and the results show that neither kind of defects have a significant effect on the final segmentation result. Updating the seed areas can effectively correct location deviation, and the CNN model can be trained with incomplete proposals caused by partial absence.

As will be understood, in further embodiments, the present invention can be extended to handle the segmentation of axial lumbar MRI for other spinal tissues such as muscles.

### 11. CONCLUSION AND INFERENCES DRAWN OF PRESENT INVENTION

The present invention which is implemented as a hybrid framework, which has been termed “Spine-GFlow” for ease of reference throughout this specification, for robust multi-tissue segmentation, the effectiveness and advantages which have been demonstrated with reference to in sagittal lumbar MRIs, which does not rely on any human intervention and manual annotation.

The rule-based method automatically generate the weak annotation for CNN training.

A clustering-based method may be utilised to generate the proposals by integrating multi-scale feature maps produced by the CNN model, which can produce proposals with shape details.

The anatomical prior is explicitly embedded via several rule-based proposal fine-tuning operations.

A comprehensive loss is introduced to simultaneously optimize the pixel classifications and feature distribution of feature maps generated by the CNN model, which significantly improves the efficiency of training.

Segmentation performance was quantitatively validated and compared with other state-of-the-art methods on the HKDDC dataset that contains the MRI obtained from 3 different machines, and the IVDM3Seg dataset.

The results demonstrate that the framework is comparable to a model trained with full supervision, however with the advantages including:
(a) Low capital power required,
(b) No need for quantum computer power or usage,
(c) Obviates human inter and intra variance,
(d) Not scanning machine dependent, and can be utilised with outputs from various types of scanning machines, and
(e) No human annotation is required.

The present invention has significant implications for many MRI analysis tasks, including pathology detection, 3D reconstruction for further auto-diagnosis, and 3D printing.

### 12. EXAMPLES OF IMPLEMENTATION OF PRESENT INVENTION

Medical images, for example Magnetic resonance images (MRIs), can simultaneously illustrate the 3D structures and potential pathologies of multiple tissues.

The present example is based on the MRI to generate the 3D reconstruction of multiple tissues to assist the surgeon in surgical planning.

In this example, there is used novel Artificial intelligence (AI) technology by way of neural network, for tissue, in particular for multi-tissue segmentation and slice super-resolution of MRI to generate the accurate 3D model for tissue, in particular for multiple tissue.

Optionally, there may be embed the Augmented Reality (AR) technology in the process and system for the preoperative and intraoperative guidance.

By way of example the hardware of the system contains the logical computing processer (CPU), parallel computing processer (GPU), random-access memory, and data storage (HDD/SSD).

The CPU processor performs multiple logically complex tasks of data transmission, communication, controlling, and rule-based image processing.

The GPU processor performs the computationally intensive tasks, including the AI assessment and training.

The CPU processor and GPU processor here refer to a set of devices that perform the same functions, which can be configured in the system according to the specific demand of computational power in the application scenarios with different scales.

For example, for AI client, the CPU and GPU processors can be integrated as separate chips on the motherboard, while in the AI server, which need to assess the MRI data of multiple large institutions simultaneously, thus the CPU and GPU processors may upgrade to the CPU and GPU servers for increasing demand of computational power.

The random-access memory saves the intermediate data of multiple specific MRI assessment tasks.

The specific hardware configuration of the memory of AI system can be alternates for different application scenarios. The data storage saves and archives the clinical MRI data, assessment results, and AI models.

The specific hardware configuration of the data storage can be alternates for different demand of data storage capability in the application scenarios with different scales.

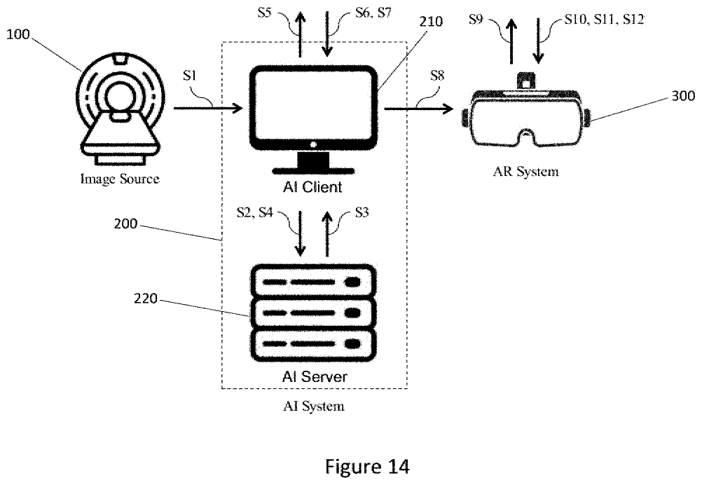

As is shown in FIG. 14, there is a schematic representation of an example of a system for use with the present invention, which includes artificial intelligence (AI) and augmented reality (AR), in which there is shown and described as follows:
100: Image Source, can be local or remote MRI equipment, which generates the raw MRI.
200: AI system.
210: AI client that transmits data, interacts with surgeon, conducts pre-processing of MRI, and generates UID for data.
220: AI server that conducts the computationally intensive task, including AI segmentation, AI slice super-resolution, 3D reconstruction.
300: AR system that provides guidance during the surgery process.
S1: Raw MRI file (Dicom file).
S2: Pre-processed MRI image generated by AI client (Dicom file).
S3: AI assessment result including segmentation and 3D reconstruction (Dicom file).
S4: AI assessment result that corrected and confirmed by surgeon (Dicom file).
S5: Signal from AI system to surgeons including the patient information, original image, and AI assessment result that is illustrated via the common display.
S6: Correction and confirmation of AI assessment result from surgeons.
S7: Surgical planning (type and position of implants) from surgeons.
S6 and S7 are a set of commands input via external devices, such as mouse and keyboard.
S8: 3D reconstruction of multiple tissues and implant proposals (OBJ file).
S1, S2, S3, S4, and S8 are transmitted via wireless communication system, such as 5G internet.
S9: Signal from AR system to surgeons that is illustrated via the head-mounted display (HMD).
S10: Optical signal from real-world.
S11: Tracker signal.
S12: Optical signal of gesture.
S10, S11, and S12 are external signals input to the AR system which are obtained via specific sensers.

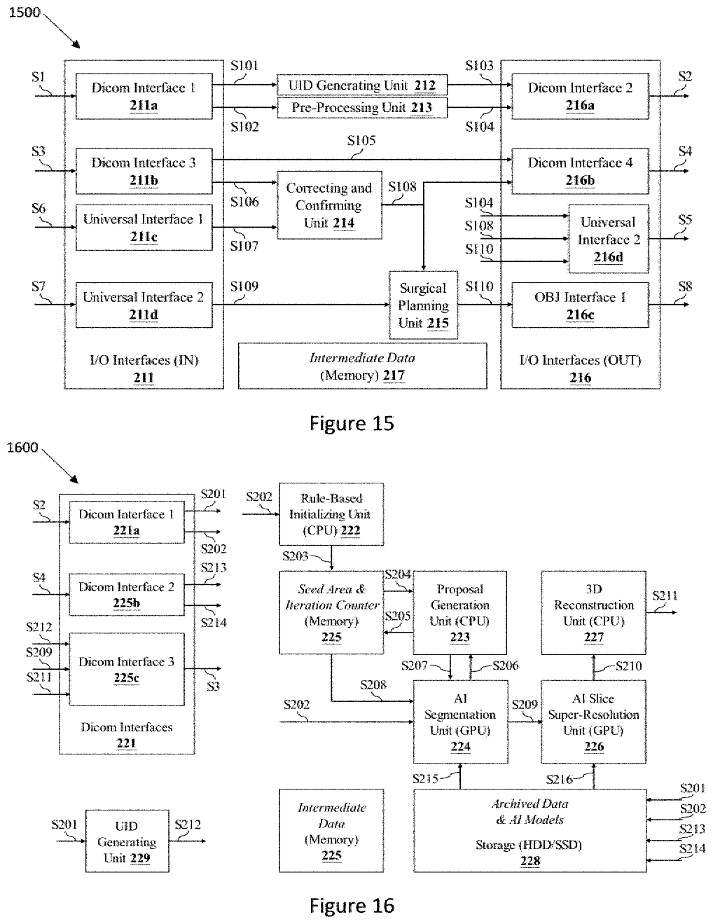

Referring to FIG. 15, there is shown a schematic representation 1500 of an example for use with the present invention of an Artificial Intelligence (AI) client 210, in which there is shown and described as follows:
211: Input/Output (I/O) interfaces (IN) that receive data and signal from surgeon.
211a: Dicom Interface 1 that receives the Dicom file from MRI source S1, reads the image data S101 and meta-data S102.
211b: Dicom Interface 3 that receives the Dicom file from AI server S3, reads the assessment result S106, and decrypts the UID S105.
211c: Universal Interface 1 that receives the correction and confirmation of AI assessment result from surgeons S6.
211d: Universal Interface 2 that receives the surgical planning from surgeons S7.
216: I/O interfaces (OUT) that output data and illustrate AI assessment result to surgeon.
216a: Dicom Interface 2 that receives the preprocessed image S104, encrypts the UID S103, and outputs a Dicom file S2 to AI server.
216b: Dicom Interface 4 that receives the confirmed AI assessment result S108, encrypts the UID S105, and outputs a Dicom file S4 to AI server.
216c: OBJ Interface 1 that output the 3D reconstruction of multiple tissues and implant proposals (surgical planning) as OBJ file S8 to AR system.
216d: Universal Interface 2 that illustrates the image, AI assessment result, 3D reconstruction from AI system to surgeons for interaction.
212: UID Generating Unit that generates the UID for image. In AI system, each image, segmentation result, 3D reconstruction, is assigned a UID, which is used for identifying, archiving, and retrieving data. The UID is defined as a character string consisting of patient ID, study date, sequential ID, instance ID, and data type (‘01’ for raw image, ‘02’ for segmentation, ‘03’ for 3D reconstruction). The UID is encrypted and decrypted with hash function by Dicom Interfaces when a Dicom file is output and received.
213: Pre-Processing Unit that conducts the pixel value normalization and denoising on the raw image.
214: Correcting and Confirming Unit that corrects the AI assessment result according to the command form surgeon.
215: Surgical Planning Unit that embeds the 3D reconstruction of implants in the 3D reconstruction of multiple tissues according to the command from surgeon.
217: Memory of AI client that save the intermediate data.
S101: MRI meta-data that contains the basic information of patient and MRI image.
S102: Raw MRI image data.
S103: UID of MRI image.
S104: Pre-processed MRI image.
S105: UID of AI assessment result.
S106: AI assessment result.
S107: Commands of correction and confirmation for AI assessment result from surgeon.
S108: Corrected and confirmed AI assessment result.
S109: Commands of surgical planning from surgeon that includes types and positions of implants.
S110: 3D reconstruction of multiple tissues and implant proposals Referring to FIG. 16 there is shown a schematic representation 1600 of an example for use with the present invention of an AI server 220, in which:
221: Dicom Interfaces that receive and output Dicom files.
221a: Dicom Interface 1 that receives the Dicom file of MRI image S2, reads the image data S202, and decrypts the UID S201.
221b: Dicom Interface 2 that receives the Dicom file of corrected and confirmed AI assessment result S4, reads the data of assessment result S214, and decrypts the UID S213.
221c: Dicom Interface 3 that receives AI assessment results (segmentation S209 and 3D reconstruction S211), encrypts the UIDs of AI assessment results, and output the Dicom file.
222: Rule-Based Initialization Unit that generates the initial seed area S203 based on the MRI image S202.
223: Proposal Generation Unit that generates the segmentation proposal based on the seed area S204 and feature maps S206, updates the seed area and iteration counter S205, and calculates the loss S207 to optimize AI model according to the generated proposal.
224: AI Segmentation Unit that produces the feature maps S206 from the MRI image S202, and produces the multi-tissue segmentation S209 under the trigger signal of iteration counter S208. The AI model is loaded from Storage S215 and iteratively optimized for each MRI case.
223 and 224 work alternately to iteratively optimize the AI model and proposal. The iteration counter records the number of iterations, which stops the iterative optimization after a certain number of iterations and triggers the AI segmentation Unit 224 with the optimized model to produce the segmentation result.
225: Memory that saves the seed area, iteration counter, and other intermediate data.
226: AI Slice Super-resolution Unit that improves the slice resolution of segmentation results. The slice resolution of clinical MRI is low (slice thickness is large), therefore an AI model was adopted that is trained to generate intermediate slices from two adjacent MRI slices to improve the slice resolution of segmentation results.
227: 3D Reconstruction Unit that produce the 3D meshing for multiple tissues based on the segmentation result via the Marching Cubes Algorithm (MCA).
228: Storage that saves and archives the MRI image data, assessment results, and AI models.
229: UID Generating Unit that generates the UID for segmentation and 3D reconstruction based on the UID of MRI image. It replaces the data type in the UID of MRI image, ‘01’, to ‘02’ for segmentation and ‘03’ for 3D reconstruction.
S201: UID of MRI image
S212, S213: UID of AI assessment results.
S202: Image data
S203: Initial seed area
S204: Seed area
S203, S204 identify the location of each tissue.
S205: Updated seed area and iteration counting signal.
S206: Feature maps that contain the multi-scale image features of each pixel in MRI.
S207: Loss for AI model optimization.
S208: Signal to trigger AI Segmentation Unit 224 to generate multi-tissue segmentation.
S209: Multi-tissue segmentation
S210: Multi-tissue segmentation with high slice resolution.
S211: 3D reconstruction (meshing) for multiple tissues.
S214: AI assessment results that are corrected and confirmed by surgeon, and will be saved and archived in storage.
S215: AI model for segmentation
S216: AI model for slice super-resolution

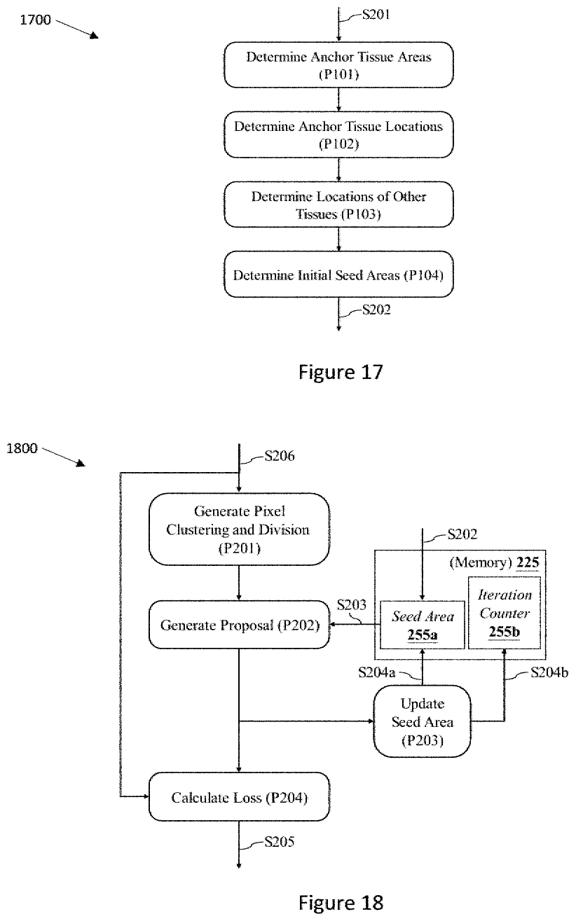

Referring to FIG. 17, there is shown a schematic representation of procedure of rule-based seed area initialization 1700 according to the present invention, in which:
P101: Determine the anchor tissue area based on the textural, shape, and location information of MRI.

The normalized and amplified image gradients, as well as shape and location selection are adopted. Anchor tissue is the most consistent and stable tissue in surgery, such as the vertebra in spinal surgery.
P102: Determine anchor tissue locations based on anchor tissue areas. It is the 3D geometric center of each anchor tissue area.
P103: Determine the locations of other tissues based on the anatomical prior knowledge about relative location to the anchor.
P104: Determine initial seed areas, which are the 3D neighbourhood centered at the location of each tissue.

Referring to FIG. 18, there is shown a schematic representation 1800 of an example of a procedure of segmentation proposal generation according to the present invention, in which:
P201: Apply a clustering-based method (k-means) on the feature maps to generate pixel clustering. Further, divide pixels into several connected components based on the pixel clustering.
P202: Select the connected components that overlap with seed area and assemble the selected connected components to generate proposal.
P203: Update the seed area as the dominate pixel cluster in each proposal, which is the pixel cluster containing the most pixel in the proposal.
P204: Calculate the loss to optimize the AI model according to the feature maps and proposal.
S204a: updated seed area.
S204b: Iteration counting signal.

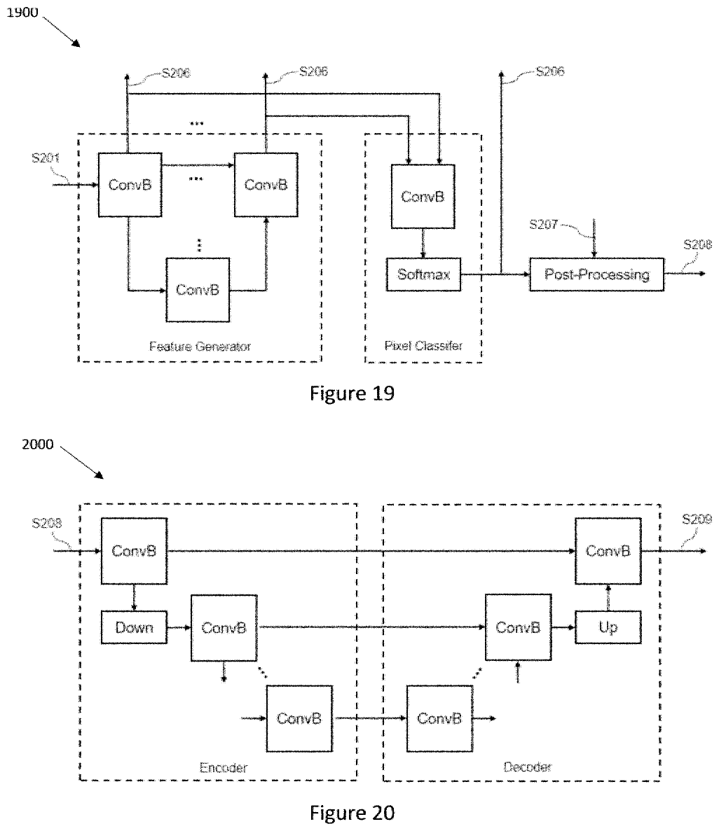

Referring to FIG. 19, there is shown a schematic representation of an example of network architecture of AI model 1900 for segmentation according to the present invention.

Feature Generator adopts the basic architecture of U-Net++, Pixel Classifier concatenates the feature maps generated by the Feature Generator and produces the pixel-wise classification that is considered as the final feature map.

Post-Processing, which consists of thresholding and 3D morphology opening, is applied on the pixel-wise classification to generate the segmentation result.

Referring to FIG. 20, there is shown a schematic representation of an example of a network architecture of AI model for slice super-resolution 2000 according to the present invention.

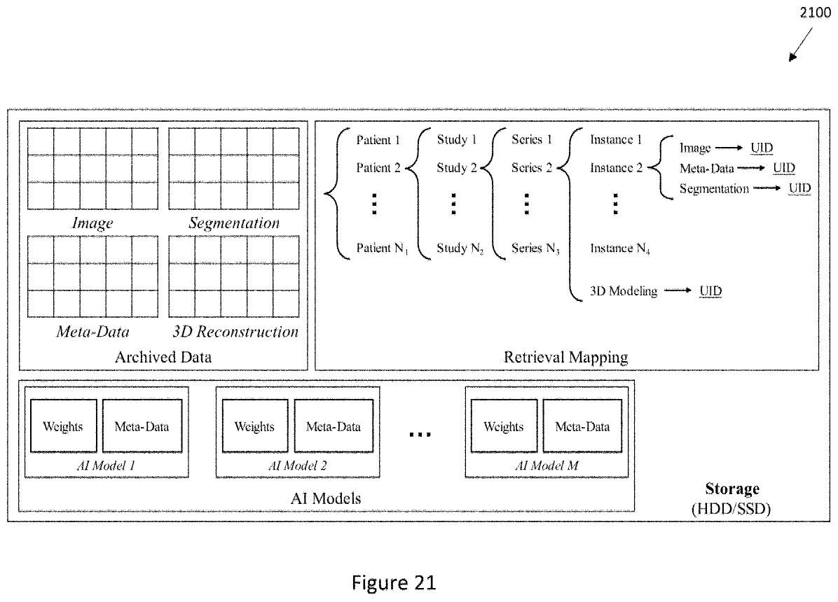

Referring to FIG. 21, there is shown a schematic representation of an example of a framework of Storage of AI system 2100, according to the present invention.

The Storage contains 3 partitions to save the archived data, retrieval mapping, and AI models.

The partition for archived data is further divided into 4 sections to save the data with different formats:

Image is saved as the matrix with the data type of float.

Segmentation is saved as the matrix with the data type of int.

Meta-data is saved as the character string.

3D Reconstruction is saved as a list containing the location and direction of each vertex and face of the meshing.

Each data points is assigned a UID.

Retrieval mapping defined the hierarchical structure and affiliation of the archived data, for the retrieving and searching the data.

UID is saved in the retrieval mapping as a proxy for the archived data.

Each AI model is saved as weights and meta-data.

Weights are the network parameters of the MRI model.

Meta-data defines the network architecture, last optimization time, samples used in the last optimization, optimizer status.

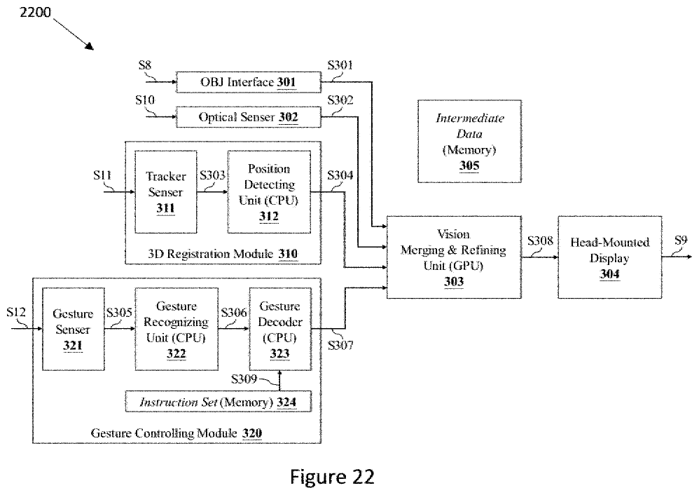

Referring to FIG. 22, there is shown a schematic representation of an example of a framework of AR system 2200, according to the present invention, in which:
301: OBJ Interface that receives the OBJ file of 3D reconstruction for surgical planning S8.
302: Optical Senser that can be the common camera and obtains the video of real-world.
310: 3D Registration Module that identifies the position of real-world tissues by detecting the position of the specific tracker attached to the target tissue. The position is utilized to register the 3D reconstruction with real-world tissues.
311: Tracker Senser that detects the position the specific tracker attached to the target tissue.
312: Position Detecting Unit that determined the position of real-word tissues according to the position of trackers. Since the shape and size of trackers is known, the tissue position can be determined by coordinate transformation.
320: Gesture Controlling Module that generates the controlling signal for AR system according to the specific gesture of surgeon.
321: Gesture Senser that can be a common camera and obtain the video of surgeon's hand movement.
322: Gesture Recognizing Unit that processes the signal from Gesture Senser and recognizes the specific gesture from it.
323: Gesture Decoder that transforms the gesture to the specific controlling signal referring the Instruction Set saved in Memory.
324: Memory that saves the Instruction Set.
303: Vision Merging & Refining Unit than merge the 3D reconstruction and real-world video according to the tissue position to generate the AR video. The Unit further refining the AR according to the gesture controlling signal from surgeon.

Surgeon can adjust the transparency of 3D model, and display mode (3D meshing, point clouds, etc.) of 3D reconstruction, or highlight the implant via the gesture controlling.
304: Head-Mounted Display that presents the AR video to surgeon.
305: Memory that saves the intermediate data.
S301: OBJ file of 3D reconstruction
S302: Real-world video
S303: Tracker position
S304: Tissue position
S305: Video of surgeon's hand movement.
S306: Gesture signal.
S307: Controlling signal.
S308: Refined AR video.
S309: Gesture-Controlling Reference

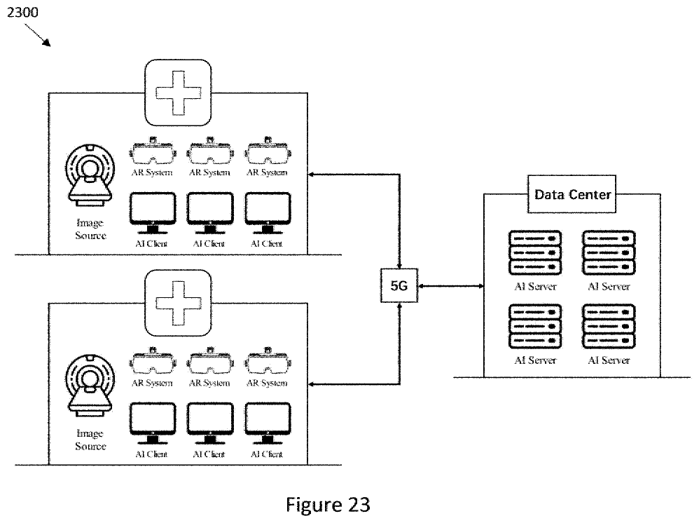

Referring to FIG. 23, there is shown a schematic representation of an example of a deployment scenarios 2300, according to the present invention.

Each hospital may equip multiple AI Clients and AR Systems.

Each clinic may equip an AI Client that can be a common office computer.

Each operating room may equip an AR System.

The clinical MRI data from multiple institutions is transferred to a regional data center, which equips multiple AI Servers to provide AI based MRI assessment and returns the assessment result to hospitals. The 5G internet is adopted for data transformation.

### 13. REFERENCES

Bearman, A., Russakovsky, O., Ferrari, V., Fei-Fei, L., 2016. What's the point: Semantic segmentation with point supervision, European Conference on Computer Vision (ECCV). Springer, pp. 549-565.
Benneker, L. M., Heini, P. F., Anderson, S. E., Alini, M., Ito, K., 2005. Correlation of radiographic and MRI parameters to morphological and biochemical assessment of intervertebral disc degeneration. European Spine Journal 14, 27-35.
Carballido-Gamio, J., Belongie, S. J., Majumdar, S., 2004. Normalized cuts in 3-D for spinal MRI segmentation. IEEE transactions on medical imaging 23, 36-44.
Cheng, C. P., Halchenko, Y. O., 2020. A new virtue of phantom MRI data: explaining variance in human participant data. F1000Research 9.
Cheung, P. W. H., Fong, H. K., Wong, C. S., Cheung, J. P. Y., 2019. The influence of developmental spinal stenosis on the risk of re-operation on an adjacent segment after decompression-only surgery for lumbar spinal stenosis. The bone & joint journal 101, 154-161.
Dai, J., He, K., Sun, J., 2015. Boxsup: Exploiting bounding boxes to supervise convolutional networks for semantic segmentation, IEEE International Conference on Computer Vision (ICCV), pp. 1635-1643.
Egger, J., Kapur, T., Dukatz, T., Kolodziej, M., Zukid, D., Freisleben, B., Nimsky, C., 2012. Square-cut: a segmentation algorithm on the basis of a rectangle shape. PloS one 7, e31064.
Harb, R., Knobelreiter, P., 2021. InfoSeg: Unsupervised Semantic Image Segmentation with Mutual Information Maximization. arXiv preprint arXiv:2110.03477.
He, X., Zhang, H., Landis, M., Sharma, M., Warrington, J., Li, S., 2017. Unsupervised boundary delineation of spinal neural foramina using a multi-feature and adaptive spectral segmentation. Medical image analysis 36, 22-40.
Hwang, J.-J., Yu, S. X., Shi, J., Collins, M. D., Yang, T.-J., Zhang, X., Chen, L.-C., 2019. Segsort: Segmentation by discriminative sorting of segments, IEEE International Conference on Computer Vision (ICCV), pp. 7334-7344.
Jensen, M. C., Brant-Zawadzki, M. N., Obuchowski, N., Modic, M. T., Malkasian, D., Ross, J. S., 1994. Magnetic resonance imaging of the lumbar spine in people without back pain. New England Journal of Medicine 331, 69-73.
Kervadec, H., Dolz, J., Tang, M., Granger, E., Boykov, Y., Ayed, I. B., 2019. Constrained-CNN losses for weakly supervised segmentation. Medical image analysis 54, 88-99.
Kervadec, H., Dolz, J., Wang, S., Granger, E., Ayed, I. B., 2020. Bounding boxes for weakly supervised segmentation: Global constraints get close to full supervision, Medical Imaging with Deep Learning. PMLR, pp. 365-381.
Khoreva, A., Benenson, R., Hosang, J., Hein, M., Schiele, B., 2017. Simple does it: Weakly supervised instance and semantic segmentation, IEEE Conference on Computer Vision and Pattern Recognition (CVPR), pp. 876-885.
Kuang, X., Cheung, J. P. Y., Wu, H., Dokos, S., Zhang, T., 2020. MRI-SegFlow: a novel unsupervised deep learning pipeline enabling accurate vertebral segmentation of MRI images, 2020 42nd Annual International Conference of the IEEE Engineering in Medicine & Biology Society (EMBC). IEEE, pp. 1633-1636.
Kulharia, V., Chandra, S., Agrawal, A., Torr, P., Tyagi, A., 2020. Box2seg: Attention weighted loss and discriminative feature learning for weakly supervised segmentation, European Conference on Computer Vision (ECCV). Springer, pp. 290-308.
Lai, M. K. L., Cheung, P. W. H., Samartzis, D., Karppinen, J., Cheung, K. M. C., Cheung, J. P. Y., 2021a. Clinical implications of lumbar developmental spinal stenosis on back pain, radicular leg pain, and disability. The Bone & Joint Journal 103, 131-140.
Lai, M. K. L., Cheung, P. W. H., Samartzis, D., Karppinen, J., Cheung, K. M. C., Cheung, J. P. Y., 2021b. The profile of the spinal column in subjects with lumbar developmental spinal stenosis. The Bone & Joint Journal 103, 725-733.
Lee, J., Yi, J., Shin, C., Yoon, S., 2021. BBAM: Bounding box attribution map for weakly supervised semantic and instance segmentation, IEEE Conference on Computer Vision and Pattern Recognition (CVPR), pp. 2643-2652.
Lin, D., Dai, J., Jia, J., He, K., Sun, J., 2016. Scribblesup: Scribble-supervised convolutional networks for semantic segmentation, IEEE Conference on Computer Vision and Pattern Recognition (CVPR), pp. 3159-3167.
Lu, J.-T., Pedemonte, S., Bizzo, B., Doyle, S., Andriole, K. P., Michalski, M. H., Gonzalez, R. G., Pomerantz, S. R., 2018. Deep Spine: Automated lumbar vertebral segmentation, disc-level designation, and spinal stenosis grading using deep learning, Machine Learning for Healthcare Conference. PMLR, pp. 403-419.
Michopoulou, S. K., Costaridou, L., Panagiotopoulos, E., Speller, R., Panayiotakis, G., Todd-Pokropek, A., 2009. Atlas-based segmentation of degenerated lumbar intervertebral discs from MR images of the spine. IEEE transactions on Biomedical Engineering 56, 2225-2231.
Mirsadeghi, S. E., Royat, A., Rezatofighi, H., 2021. Unsupervised image segmentation by mutual information maximization and adversarial regularization. IEEE Robotics and Automation Letters 6, 6931-6938.
Neubert, A., Fripp, J., Engstrom, C., Schwarz, R., Lauer, L., Salvado, O., Crozier, S., 2012. Automated detection, 3D segmentation and analysis of high resolution spine MR images using statistical shape models. Physics in Medicine & Biology 57, 8357.
Pathak, D., Krahenbuhl, P., Darrell, T., 2015. Constrained convolutional neural networks for weakly supervised segmentation, IEEE International Conference on Computer Vision (ICCV), pp. 1796-1804.
Pfirrmann, C. W. A., Metzdorf, A., Zanetti, M., Hodler, J., Boos, N., 2001. Magnetic resonance classification of lumbar intervertebral disc degeneration. Spine 26, 1873-1878.
Qu, H., Wu, P., Huang, Q., Yi, J., Yan, Z., Li, K., Riedlinger, G. M., De, S., Zhang, S., Metaxas, D. N., 2020. Weakly supervised deep nuclei segmentation using partial points annotation in histopathology images. IEEE Transactions on Medical Imaging 39, 3655-3666.
Rajchl, M., Lee, M. C. H., Oktay, O., Kamnitsas, K., Passerat-Palmbach, J., Bai, W., Damodaram, M., Rutherford, M. A., Hajnal, J. V., Kainz, B., 2016. Deepcut: Object segmentation from bounding box annotations using convolutional neural networks. IEEE transactions on medical imaging 36, 674-683.
Ronneberger, O., Fischer, P., Brox, T., 2015. U-net: Convolutional networks for biomedical image segmentation, International Conference on Medical Image Computing and Computer-Assisted Intervention (MICCAI). Springer, pp. 234-241.
Samartzis, D., Karppinen, J., Chan, D., Luk, K. D. K., Cheung, K. M. C., 2012. The association of lumbar intervertebral disc degeneration on magnetic resonance imaging with body mass index in overweight and obese adults: a population-based study. Arthritis & Rheumatism 64, 1488-1496.
Song, C., Huang, Y., Ouyang, W., Wang, L., 2019. Box-driven class-wise region masking and filling rate guided loss for weakly supervised semantic segmentation, IEEE Conference on Computer Vision and Pattern Recognition (CVPR), pp. 3136-3145.
Tang, M., Djelouah, A., Perazzi, F., Boykov, Y., Schroers, C., 2018. Normalized cut loss for weakly-supervised cnn segmentation, IEEE Conference on Computer Vision and Pattern Recognition (CVPR), pp. 1818-1827.
Valvano, G., Leo, A., Tsaftaris, S. A., 2021. Learning to Segment from Scribbles using Multi-scale Adversarial Attention Gates. IEEE Transactions on Medical Imaging.
Van Gansbeke, W., Vandenhende, S., Georgoulis, S., Van Gool, L., 2021. Unsupervised semantic segmentation by contrasting object mask proposals. arXiv preprint arXiv:2102.06191.
Yoo, I., Yoo, D., Paeng, K., 2019. Pseudoedgenet: Nuclei segmentation only with point annotations, International Conference on Medical Image Computing and Computer-Assisted Intervention (MICCAI). Springer, pp. 731-739.
Zhou, Z., Siddiquee, M. M. R., Tajbakhsh, N., Liang, J., 2019. Unet++: Redesigning skip connections to exploit multiscale features in image segmentation. IEEE transactions on medical imaging 39, 1856-1867.

1. A process for tissue modelling from a medical image of a subject, for forming a three-dimensional (3D) model of a region of interest (ROI) of a subject with one or more tissue types, said process including the steps of:
(i) utilising a rule-based method that automatically generates the weak annotation, initial seed area from a medial image;
(ii) utilising a proposal generation method that integrates the multi-scale image features and anatomical prior; and
(iii) a comprehensive loss for CNN training that optimizes the pixel classification and feature distribution simultaneously.

2. A process for tissue modelling of a subject of one or more tissue type at a region of interest (ROI) of said subject, for forming a three-dimensional (3D) model of a region of interest (ROI) of a subject with one or more tissue types, said process including the steps of:
(i) utilising a rule-based method to automatically generate a weak annotation from a midline slice of a subject in a 3 Dimensional (3D) medical image of said subject, wherein the rule-based method detects the approximate tissue locations from the midline slice, and, and further determines the initial seed areas;
(ii) developing a neural network model to generate multi-scale feature maps and pixel classifications from the midline slice;
(iii) utilising a clustering-based method to generate segmentation proposals based on multi-scale feature maps and the seed areas from (i) and (ii).
(iv) further fine-tuning the proposal with several rule-based operations to explicitly embed within an anatomical prior of the region of interest (ROI), and wherein the seed areas are updated according to the fine-tuned proposal; and
(v) training the neural network model with a comprehensive loss, which simultaneously optimizes the pixel classification and feature distribution of feature maps based on the proposals.

3. The process according to claim 2, wherein the process provides for modelling of multiple tissue type of a subject in the region of interest (ROI).

4. The process according to claim 2, wherein the medical image is a Magnetic Resonance Imaging (MRI) image.

5. The process according to claim 2, further including the steps of:
utilising one of more further slices of said subject acquired of the region of interest (ROI) of the subject at varying depths within the region of interest (ROI) and simultaneously optimizes the pixel classification and feature distribution of feature maps based on the proposals for said one or more further slices by the process of claim 2 steps (i) to (ii); and
forming a three dimensional (3D) model of the region of interest (ROI) of the tissue of the subject from the midline slice and the one or more further slices.

6. A system for tissue modelling of a subject of one or more tissue type at a region of interest (ROI) of said subject, for forming a three-dimensional (3D) model of a region of interest (ROI) of a subject with one or more tissue types, said system including an input module, a processor, a neutral network, and an output module, wherein:
said input module receives a 3 Dimensional (3D) medical image containing a plurality of 2 Dimensional (2D) slices of a subject of a region of interest (ROI) of said subject;
said processor and a neural network provide the process of:
(i) using a rule-based method to automatically generate a weak annotation from the plurality of 2 Dimensional (2D) slices of said subject, wherein the rule-based method detects the approximate tissue locations from the plurality of 2 Dimensional (2D) slices, and further determines the initial seed areas;
(ii) developing a neural network model to generate multi-scale feature maps and pixel classifications from the plurality of 2 Dimensional (2D) slices;
(iii) utilising a clustering-based method to generate segmentation proposals based on multi-scale feature maps and the seed areas;
(iv) further fine-tuning the proposal with several rule-based operations to explicitly embed within an anatomical prior of the region of interest (ROI), and wherein the seed areas are updated according to the fine-tuned proposal; and
(i) training the neural network model with a comprehensive loss, which simultaneously optimizes the pixel classification and feature distribution of feature maps based on the proposals; and
said output module provides an output representation a three-dimensional (3D) model of a region of interest (ROI) of the subject with one or more tissue types.

7. A Three-dimensional (3D) medical model of a region of interest of a subject, wherein the three-dimensional medical model is formed by way of the process of claim 5.

8. A process for providing a three-dimensional (3D) tissue model of one or more tissues or tissue types, the process including the steps of:
(i) providing unique rule for a seed, that is a portion of an analytical site which is used to start, in a medical image;
(ii) using the seed area to determine a segmentation proposal and
(iii) training a deep learning model with the segmentation proposal.
